(19) United States
(12) Patent Application Publication
(10) Pub. No.: US 2018/0236080 A1
(43) Pub. Date: Aug. 23, 2018

(54) FAST-ACTING INSULIN COMPOSITION COMPRISING A CITRIC ACID SALT

(71) Applicant: ADOCIA, Lyon (FR)

(72) Inventor: Gerard SOULA, Meyzieu (FR)

(73) Assignee: ADOCIA, Lyon (FR)

(21) Appl. No.: 15/625,684

(22) Filed: Jun. 16, 2017

(30) Foreign Application Priority Data
Feb. 22, 2017 (FR) 17/51394

(51) Int. Cl. A61K 47/12; A61K 38/28; A61K 9/00; A61K 47/26

(52) U.S. Cl. CPC A61K 47/12; A61K 47/26; A61K 9/0019; A61K 38/28

(57) ABSTRACT

In embodiments, the present disclosure provides an aqueous pharmaceutical composition that includes insulin in hexameric form, and citric acid or a salt thereof at a concentration from 6 to 30 mM, wherein the composition is suitable for injecting into a diabetic patient to treat diabetes.

The present disclosure relates to a fast-acting insulin composition.

### BACKGROUND

Since the production of insulin by genetic engineering, in the early 1980s, diabetic patients benefit from human insulin to treat themselves. This product greatly improves this therapy since the immunological risks associated with the use of non-human insulin, especially from pigs, are eliminated. However, human insulin injected subcutaneously has a hypoglycemic effect only after 60 minutes, which implies that diabetic patients treated with human insulin should inject themselves 30 minutes before meals.

One of the problems to be solved in order to improve the health and comfort of diabetic patients is to provide insulin formulations that show a faster hypoglycemic response than human insulin and, if possible approach the physiological response of the healthy person. Endogenous insulin secretion in healthy individuals is immediately triggered by increased blood sugar levels. The goal is to reduce as much as possible the time lapse between the injection of insulin and the beginning of the meal.

Today, it is recognized that the provision of such formulations is useful so that the management of the disease is the best possible.

Genetic engineering has made it possible to provide a solution with the development of fast-acting insulin analogs. These insulins are modified on one or two amino acids so as to be absorbed faster in the bloodstream after a subcutaneous injection. These include insulin lispro (Humalog®, Lilly) or insulin aspart (Novolog®/Novorapid®, Novo Nordisk) which are stable aqueous insulin solutions with hypoglycemic responses faster than that of human insulin. Therefore, patients treated with one of these fast-acting insulins may inject insulin only 15 minutes before the meal.

The general principle of fast-acting insulin analogs is to form hexamers at a concentration of 100 IU/ml in order to ensure the stability of insulin in the commercial product while promoting the very rapid dissociation of these hexamers into monomers after subcutaneous injection in order to accelerate the action.

Typically, human insulin, as formulated in its commercial form, does not allow obtaining, in terms of kinetics, a hypoglycemic response close to the physiological response generated by the beginning of a meal (increase in glycemia), because, at the customary concentration (100 IU/ml), in the presence of zinc and other excipients, it self-associates as a hexamer while it is active in the form of a monomer and dimer. Human insulin is prepared in the form of hexamers in order to be stable for almost 2 years at 4° C. In the form of monomers, it has a very high propensity to aggregate and then to fibrillate, which causes it to lose its activity. Moreover, in this aggregated form, it presents an immunological risk for the patient.

The dissociation of human insulin hexamers into dimers and dimers into monomers delay its action by nearly 20 minutes compared to a fast-acting insulin analog (Brange J. et al., Advanced Drug Delivery Review, 35, 1999, 307-335).

Moreover, the kinetics of the passage of the insulin analogs into the bloodstream, as well as their kinetics of reduction of the glycemia, are not always optimal and there is a real need for a formulation having an even faster action time in order to approximate the kinetics of endogenous insulin secretion in healthy people.

### SUMMARY

Surprisingly, however, the applicant has succeeded in developing formulations capable of accelerating insulin, i.e., the passage of the insulin into the bloodstream and/or the decrease of the level of glucose in the blood, by using citric acid or a salt thereof only.

The hexameric nature of the insulin is not affected by the inclusion of citric acid or a salt thereof, as is confirmed by the examples of the association state of insulin lispro or insulin aspart measured by circular dichroism in the presence of citric acid or a salt thereof.

In the present disclosure, the various problems described above are solved, in whole or in part, since it may allow in particular for the production of a formulation of insulin capable, after administration, of accelerating the passage of the insulin into the bloodstream and/or reducing blood glucose more rapidly compared to the corresponding commercial insulin products, while leading to compositions having a physical and/or chemical stability upon storage.

### BRIEF DESCRIPTION OF THE DRAWINGS

FIG. 1: DGlucose (% of the basal value) as a function of time after injection (min.). The curve plotted with the squares corresponds to Example IA4, and the curve plotted with the triangles corresponds to Example IA1.

FIG. 2: DInsulin (pM/L) as a function of time after injection (min.). The curve plotted with the squares corresponds to Example IA4, and the curve plotted with the triangles corresponds to Example IA1.

FIG. 3: DGlucose (% of the basal value) as a function of time after injection (min.). The curve plotted with the squares corresponds to Example IA9, and the curve plotted with the triangles corresponds to Example IA1.

FIG. 4: DInsulin (pM/L) as a function of time after injection (min.). The curve plotted with the squares corresponds to Example IA9, and the curve plotted with the triangles corresponds to Example IA1.

FIG. 5: describes on the x-axis, from left to right:
A: insulin lispro 100 IU/ml (Example I.A1),
B: insulin lispro 100 IU/ml and sodium citrate at 9.3 mM (Example I.A4),
C: insulin lispro 100 IU/ml and sodium citrate at 18.6 mM (Example I.A9),
D: insulin lispro 100 IU/ml and EDTA at 300 μM (Example I.A14)
   and on the y-axis the CD signal at 240 nm (deg·cm²·dmol⁻¹).

FIG. 6: describes on the x-axis, from left to right:
A: A: insulin lispro 100 IU/ml (Example I.A1),
B: insulin lispro 100 IU/ml, sodium citrate at 9.3 mM and Polysorbate at 8 μM (Example I.A20),
   and on the y-axis the CD signal at 240 nm (deg·cm2·dmol−1).

FIG. 7: DGlucose (% of the basal value) as a function of time after injection (min.). The curve plotted with the squares corresponds to Example IIA4, and the curve plotted with the triangles corresponds to Example IIA1.

FIG. 8: DInsulin (pM/L) as a function of time after injection (min.). The curve plotted with the squares corresponds to Example IIA4, and the curve plotted with the triangles corresponds to Example IIA1.

FIG. 9: describes on the x-axis, from left to right:
A: insulin aspart 100 U/ml (Example II.A1),
B: insulin aspart 100 U/ml and sodium citrate at 9.3 mM (Example II.A4),
C: insulin aspart 100 U/ml and sodium citrate at 18.6 mM (Example II.A9),
D: insulin aspart 100 U/ml and EDTA at 300 μM (Example II.A14),
   and on the y-axis the CD signal at 240 nm (deg·cm2·dmol−1).

FIG. 10: describes on the x-axis, from left to right:
A: insulin aspart 100 IU/ml (Example II.A1),
B: insulin aspart 100 IU/ml, sodium citrate at 9.3 mM and Polysorbate at 8 μM (Example II.A20),
   and on the y-axis the CD signal at 240 nm (deg·cm2·dmol−1).

### DETAILED DESCRIPTION

In embodiments, the composition comprises insulin in hexameric form, and citric acid or a salt thereof.

In embodiments, the composition comprises insulin in hexameric form, and citric acid or a salt thereof at a concentration from 6 to 30 mM.

In embodiments, the composition is in the form of an aqueous solution.

In embodiments, the composition is an aqueous pharmaceutical composition.

In embodiments, the composition is an aqueous pharmaceutical composition suitable for injecting into a diabetic patient to treat diabetes.

The diabetes treated by the compositions herein may be, for example type 1 diabetes and/or type 2 diabetes.

Thus in the disclosure “composition” can mean any of the above type of compositions.

In embodiments, the composition does not contain any one of EDTA, anionic compounds whose chemical structure includes at least one carboxyl functional group to which is covalently bonded an amino-acid residue, saccharides whose chemical structure includes at least one carboxyl functional group to which is covalently bonded an amino-acid residue, and oligosaccharides whose chemical structure includes at least one carboxyl functional group to which is covalently bonded an amino-acid residue.

In embodiments, the present disclosure provides a method of treating diabetes comprising administering to a human in need thereof an effective dose of a composition such as defined herein.

In embodiments, the present disclosure provides a method preparing a composition such as defined herein.

In embodiments, the present disclosure provides an injection device comprises a composition such as defined herein.

In embodiments, the present disclosure provides a closed and/or sealed container containing a composition such as defined herein

In embodiments, the present disclosure provides a vial containing a composition such as defined herein.

In embodiments, the present disclosure provides a cartridge containing a composition such as defined herein.

In embodiments, the present disclosure provides a pump containing a composition such as defined herein.

In embodiments, the present disclosure provides a kit comprising the composition and instructions explaining how to use it.

When used herein, “insulin” means prandial insulin, such as human insulin, or rapid-acting insulin, such as an analog, structural variant or mutant of human insulin that has the functional activity of human insulin but has a faster onset of action than human insulin. Known rapid-acting analogs of human insulin include insulin lispro and insulin aspart. In embodiments, the insulin employed in the present compositions is in hexameric form, or substantially in hexameric form, such as at least 80%, 85%, 90%, 95%, 97%, 98%, or 99% in hexameric form. In other embodiments, the insulin employed in the present compositions has less in hexameric form, such as 50%, 40%, 30%, 20%, or 10% or less.

The term “insulin analog” means a recombinant insulin whose primary sequence contains at least one modification relative to the primary sequence of human insulin.

In embodiments, the insulin analog is either the insulin lispro (Humalog®) or the insulin aspart (Novolog®, Novorapid®).

In embodiments, the insulin analog is the insulin lispro (Humalog®).

In embodiments, the insulin analog is the insulin aspart (Novolog®, Novorapid®). In embodiments, the present disclosure provides a composition, characterized in that its onset of action in humans is at least 5%, 10%, 15%, 30%, 50% or 70% lower, i.e., more rapid, than that of a reference composition at the same insulin concentration in the absence of citric acid or a salt thereof.

In embodiments, the present disclosure provides a composition, characterized in that its onset of action in humans is at most 90% or 80% lower, i.e., more rapid, than that of the reference composition at the same insulin concentration in the absence of citric acid or a salt thereof.

In embodiments, the present disclosure provides a composition, characterized in that its onset of action in humans is at least 5%, 10%, 15%, 30%, 50% or 70% lower, i.e., more rapid, than that of the reference composition at the same insulin concentration in the absence of citric acid or a salt thereof and at most 90% or 80% lower than that of the reference composition at the same insulin concentration in the absence of citric acid or a salt thereof.

The phrase “citric acid or a salt thereof” as used herein refers to pharmaceutically acceptable salts of citric acid, such as sodium citrate, potassium citrate, calcium citrate, and magnesium citrate, particularly sodium citrate. Included are mono-, di-, and tri-cation salts of citric acid, such as trisodium citrate. Although not always expressly stated, embodiments described herein contemplate the use of citric acid, a citric acid salt, and mixtures thereof, including mixtures of different citric acid salts.

By “aqueous solution” is meant a solution within the meaning of the European Pharmacopoeia.

The solution thus desirably complies with the European Pharmacopoeia 8.0, which defines that the characteristics exhibited by an injectable preparation of soluble insulin as including that it is a colorless, non-opalescent liquid, and free of foreign substances; traces of very fine sediments may be deposited during storage (01/2008: 0834).

The solution may be a non-opalescent or even clear liquid.

According to Chapter 2.2.1 of the European Pharmacopoeia 8.0, a liquid is considered to be clear when it has an opalescence which is not more pronounced than that of the control suspension, which has an opalescence value of 3 NTU. The opalescence of the solution can be determined by the visual method and/or by the instrumental method, called turbidimetry. Said methods are defined in Chapter 2.2.1 of the European Pharmacopoeia 8.0.

In embodiments, the solution has a turbidity of less than or equal to 3 NTU according to the different methods described in Chapter 2.2.1. of the European Pharmacopoeia 8.0.

In embodiments, the compositions are sterile compositions, such as wherein sterilization is by known methods. In some embodiments, sterilization is by filtration on a 0.22 μm membrane, for example by filtration on a SLGV033RS membrane, Millex-GV from Millipore, a 0.22 μm PVDF membrane.

In embodiments, the composition is an aqueous pharmaceutical composition suitable for injecting into a diabetic patient to treat diabetes.

By “aqueous pharmaceutical composition which is suitable for injection” is meant a composition complying with the USP and/or European pharmacopeia, including with respect to sterility. In particular, in terms of sterility, the composition may comply with USP 39<71> and/or European pharmacopeia 9.0, in 2.6.1.

In embodiments, the present disclosure provides an aqueous pharmaceutical composition suitable for injecting into a diabetic patient to treat diabetes with an onset of action in humans that is at least 5%, 10%, 15%, 30%, 50% or 70% more rapid than that of the reference composition at the same insulin concentration in the absence of citric acid or a salt thereof.

In embodiments, the onset of action is measured with some pharmacodynamics parameters relevant for time action, such as the time for the minimum glucose level in the blood and/or the time to reach 50% of the minimum level in the blood (respectively Tmin glucose and T50% Rmin glucose). The T50% Rmin glucose is the time to reach 50% of the minimum level of glucose in the blood, also called Rmin.

The T50% Rmin glucose is estimated by linear interpolation.

In embodiments, the onset of action is the time to reach 50% of the minimum level in the blood, T50% Rmin glucose.

Unless otherwise specified herein, T50% means early T50%.

In embodiments, the present disclosure provides a composition, characterized in that its onset of appearance, i.e., of appearance of insulin in the blood, in humans is at least 5%, 10%, 15%, 30%, 50% or 70% more rapid than that of a reference composition at the same insulin concentration in the absence of citric acid or a salt thereof.

In embodiments, the present disclosure provides a composition, characterized in that its onset of appearance, i.e., of appearance of insulin in the blood, in humans is at most 95%, 90%, or 85% more rapid than that of the reference composition at the same insulin concentration in the absence of citric acid or a salt thereof.

In embodiments, the present disclosure provides a composition, characterized in that its onset of appearance, i.e., of appearance of insulin in the blood, in humans is at least 5%, 10%, 15%, 30%, 50% or 70% more rapid than that of the reference composition at the same insulin concentration in the absence of citric acid or a salt thereof and at most 95%, 90%, or 85% more rapid than that of the reference composition at the same insulin concentration in the absence of citric acid or a salt thereof.

In embodiments, the composition is an aqueous pharmaceutical composition suitable for injecting into a diabetic patient to treat diabetes.

In embodiments, the present disclosure provides an aqueous pharmaceutical composition suitable for injecting into a diabetic patient to treat diabetes with an onset of appearance in humans is at least 5%, 10%, 15%, 30%, 50% or 70% more rapid than that of a reference composition at the same insulin concentration in the absence of citric acid or a salt thereof.

In embodiments, the onset of appearance is measured with some pharmacodynamics parameters relevant to time action, such as the time to reach the maximum insulin concentration in the blood and/or the time to reach 50% of the maximum insulin concentration in the blood, respectively Tmax insulin and T50% Cmax insulin. The T50% Cmax insulin is the time to reach 50% of the maximum level of insulin in the blood, also called Cmax.

The T50% Cmax insulin is estimated by linear interpolation.

In embodiments, the onset of appearance is measured with some pharmacodynamics parameters relevant to time action, such as the time to reach the maximum insulin concentration in the blood and/or the time to reach 50% of the maximum insulin concentration in the blood (respectively Tmax insulin and T50% Cmax insulin). The T50% Cmax insulin is the time to reach 50% of the maximum level of insulin in the blood.

In embodiments, the onset of appearance is measured with the time to reach 50% of the maximum insulin concentration in the blood, T50% Cmax insulin.

In embodiments, the present disclosure provides a composition characterized in that it improves (i.e., increases) the fast uptake of insulin in humans by at least 10%, 30%, 50%, 70% or 90% relative to a reference composition comprising the same insulin concentration without citric acid or salt thereof.

In embodiments, the present disclosure provides a composition, characterized in that it improves the fast uptake of insulin in humans by at most 95%, or 90% relative to a reference composition comprising the same insulin concentration without citric acid or salt thereof.

In embodiments, the present disclosure provides a composition characterized in that it improves the fast uptake of insulin in humans by at least 10%, 30%, 50%, 70% or 90% relative to a reference composition comprising the same insulin concentration without citric acid or salt thereof and at most 95%, or 90% relative to a reference composition comprising the same insulin concentration without citric acid or salt thereof.

In embodiments, the composition is an aqueous pharmaceutical composition suitable for injecting into a diabetic patient to treat diabetes.

In embodiments, the present disclosure provides an aqueous pharmaceutical composition suitable for injecting into a diabetic patient to treat diabetes improving the fast uptake of insulin in humans by at least 10%, 30%, 50%, 70% or 90% relative to a reference composition comprising the same insulin concentration without citric acid or salt thereof.

In embodiments, the improvement of fast uptake of insulin is measured with pharmacodynamics parameters relevant to time action, such as the early partial area under the serum insulin concentration from time 0 to time 15 minutes and/or from time 0 to time 30 minutes (respectively AUC-Ins0-15 and AUC-Ins0-30).

In embodiments, the improvement of fast uptake of insulin is measured with the early partial area under the serum insulin concentration from time 0 to time 30 minutes, AUC-Ins0-30.

In embodiments, the improvement of fast uptake of insulin is accomplished in a method of treatment comprising administering to a patient in need thereof a composition described herein.

The protocol for the measurement of pharmacodynamics and pharmacokinetics of insulin solutions in humans is as follows:

Each subject (healthy or with type 1 or type 2 diabetes) is randomly allocated to one of the treatment group (reference or tested insulin products). The insulin products are administered by subcutaneous injection into a lifted skin fold (generally of the abdominal wall, the thigh or the arm). Blood samples for determination of serum (or plasma) insulin are taken at predefined time points (every 4 or 5 minutes within the first hour post-dose is commonly used for fast insulin products). Insulin concentrations in serum (or plasma) are determined using an appropriate method (ELISA, RIA). The serum insulin concentration profiles and actual times are used to calculate one or more of the following pharmacokinetic parameters:
Cmax insulin corresponding to the maximum serum (or plasma) insulin concentration,
Tmax insulin corresponding to the time to reach the maximum serum (or plasma) insulin concentration,
Early T50% Cmax insulin corresponding to the time to reach 50% of the maximum serum (or plasma) insulin concentration before Tmax,
Late T50% Cmax insulin corresponding to the time to reach 50% of the maximum serum (or plasma) insulin concentration after Tmax,
AUC-Ins0-15 min corresponding to the early partial areas under the serum (or plasma) insulin curve from time 0 to time 15 minutes post-administration,
AUC-Ins0-30 min corresponding to the early partial areas under the serum (or plasma) insulin curve from time 0 to time 30 minutes post-administration,
AUC-Ins60-last corresponding to the late partial areas under the serum (or plasma) insulin curve from time 60 minutes to the last valid measurement time post-administration, and/or
AUC-Ins120-last corresponding to the late partial areas under the serum (or plasma) insulin curve from time 120 minutes to the last valid measurement time post-administration.

Pharmacokinetic parameters are determined based on standard non-compartmental methods using Phoenix WinNonlin. Early T50% Cmax insulin and Tmax insulin are commonly used to evaluate the onset of exposure of insulin formulations. AUC-Ins0-15 min and AUC-Ins0-30 min are commonly used to evaluate the early exposure of insulin formulations. Late T50% Cmax insulin is commonly used to evaluate the offset of exposure of insulin formulations. AUC-Ins60-last and AUC-Ins120-last are commonly used to evaluate the late exposure of insulin formulations. Early and Late T50% Cmax insulin are estimated by linear interpolation.

Such measures could be done on relevant animal model for assessing the pharmacokinetics of insulin as pig models. In the case of a pig model the protocol for the measurement of pharmacodynamics and pharmacokinetics of insulin solutions is as follows:

Domestic pigs weighing approximately 50 kg, previously catheterized at the jugular, are fasted 2.5 hours before the start of the experiment. In the hour before the injection of insulin, 3 blood samples are taken to determine the basal level of glucose and insulin.

Injection of insulin at a dose of 0.125 IU/kg for insulin aspart is performed subcutaneously in the flank of the animal using an insulin pen (Novo, Sanofi or Lilly) equipped with a 31 G needle.

Blood samples are then taken every 4 minutes for 20 minutes and then every 10 minutes for up to 3 hours. After each sampling, the catheter is rinsed with a dilute solution of heparin.

A drop of blood is taken to determine glycemia by means of a glucose meter.

The pharmacodynamic curves of the glucose expressed as a percentage of the basal level were then plotted. The following pharmacodynamic endpoint were determined based on standard non-compartmental methods using Phoenix WinNonlin:
Tmin glucose corresponding to the times to the minimum glucose level in the blood,
Early T20% Rmin glucose corresponding to the times to 20% of the minimum glucose level in the blood before Tmin,
Early T50% Rmin glucose corresponding to the times to 50% of the minimum glucose level in the blood before Tmin, and
AUC-BG0-30 min corresponding to the early partial areas below the baseline and above the glucodynamic response curve from time 0 to time 30 minutes.

Early T50% Rmin glucose and Tmin glucose are commonly used to evaluate the onset of glucose-lowering effect of insulin formulations. AUC-BG0-30 min is commonly used to evaluate the early glucose lowering effect of insulin formulations. Early T20% Rmin glucose and early T50% Rmin glucose were estimated by linear interpolation.

The remaining blood was collected in a dry tube and centrifuged to isolate the serum. Insulin levels in serum samples were measured by the sandwich ELISA (Enzyme-Linked ImmunoSorbent Assay) method for each pig.

The pharmacokinetic curves expressed in delta of the basal level were then plotted. The following pharmacokinetic parameters were determined based on standard non-compartmental methods using Phoenix WinNonlin:
Tmax insulin corresponding to the time to reach the maximum serum insulin concentration,
Early T20% Cmax insulin corresponding to the time to reach 20% of the maximum serum insulin concentration before Tmax,
Early T50% Cmax insulin corresponding to the time to reach 50% of the maximum serum insulin concentration before Tmax,
AUC-Ins0-15 min corresponding to the early partial areas under the serum insulin curve from time 0 to time 15 minutes post-administration, and
AUC-Ins0-30 min corresponding to the early partial areas under the serum insulin curve from time 0 to time 30 minutes post-administration.

Early T50% Cmax insulin and Tmax insulin are commonly used to evaluate the onset of exposure of insulin formulations. AUC-Ins0-15 min and AUC-Ins0-30 min are commonly used to evaluate the early exposure of insulin formulations. Early T20% Cmax insulin and Early T50% Cmax insulin were estimated by linear interpolation.

The pig is well known to be a relevant model for assessing the pharmacokinetics of insulin after subcutaneous dosing in human patients, as disclosed by A. Plum, in Drug Metab Dispos. 28 (2):155-160, 2000. More particularly, the pig model was used in the development of insulin aspart (NovoLog®, Novo Nordisk) and faster insulin aspart (FIAsp®, Novo Nordisk) to evaluate their primary pharmacodynamics and pharmacokinetics, see for example the Assessment report EMA/CHMP/50360/2017 section 2.3.

In embodiments, the present disclosure provides a composition in the form of an aqueous solution comprising insulin in hexameric form and at least one citric acid or a salt thereof, for use in a method for treating diabetic patients, characterized in that it improves, i.e., increases, the rapid absorption of insulin.

In embodiments, the present disclosure also provides a method for treating diabetic patients, for administering a composition in the form of an aqueous solution, comprising insulin in hexameric form and at least one citric acid or a salt thereof, characterized in that it improves, i.e., increases the rapid absorption of insulin.

In embodiments, the present disclosure provides a method for treating diabetic patients, by administering to a patient in need of the treatment a composition in the form of an aqueous solution that comprises insulin in hexameric form and at least one citric acid or a salt thereof, characterized in that its onset of action and/or of appearance in humans is at least 5%, 10%, 15%, 30%, 50% or 70% lower, more rapid and/or improvement of the early insulin uptake than that of the reference composition at the same insulin concentration in the absence of citric acid salt.

In embodiments, the composition, in the form of an aqueous solution, comprising insulin in hexameric form and at least one citric acid or a salt thereof, for use in a method for treating diabetic patients, is characterized in that its onset of action and/or of appearance in humans is at least 5%, 10%, 15%, 30%, 50% or 70% lower, more rapid, and/or improvement of the early insulin uptake than that of a reference composition at the same insulin concentration in the absence of citric acid or a salt thereof

In embodiments, the composition, in the form of an aqueous solution that comprises insulin in hexameric form and at least one citric acid or a salt thereof, for use in a method for treating diabetic patients is characterized in that its onset of action and/or of appearance in humans is at least 70% lower or more rapid than that of a reference composition at the same insulin concentration in the absence of citric acid or a salt thereof.

In some embodiments, the composition increases the area under the curve of the insulin concentration in the serum as a function of time (AUC) between 0 and 15 minutes or 0 and 30 minutes after administration (AUC-Ins0-15 or 0-30 min) by at least 10%, 30%, 50%, 70%, or 90% relative to a reference composition at the same insulin concentration in the absence of citric acid or a salt thereof.

In embodiments, the present disclosure provides a composition, in the form of an aqueous solution, comprising insulin in hexameric form and at least one citric acid or a salt thereof for use in a method for treating diabetic patients, characterized in that it improves, i.e., increases, the rapid absorption of insulin, said improvement being measured by the increase of at least 10%, 30%, 50%, 70%, or 90% of the area under the curve of the insulin concentration in the serum as a function of time (AUC) between 0 and 15 minutes or 0 and 30 minutes after administration (AUC-Ins 0-15 or 0-30 min), relative to a reference composition at the same insulin concentration in the absence of citric acid or a salt thereof.

In embodiments, the present disclosure provides a method for treating diabetic patients, for administering a composition in the form of an aqueous solution, comprising insulin in hexameric form and at least one citric acid or a salt thereof, characterized in that it allows improving, i.e., increasing, the rapid diminution of glucose in the serum.

In some embodiments, the composition increases the area below the baseline and above the glucodynamic response curve in the serum as a function of time (AUC) between 0 and 30 minutes after administration (AUC-BG 0-30 min) by at least 10%, 30%, 50%, 70%, or 90% relative to a reference composition at the same insulin concentration in the absence of citric acid or a salt thereof.

In embodiments, the present disclosure provides an aqueous pharmaceutical composition comprising:
insulin in hexameric form, and citric acid or a salt thereof at a concentration from 6 to 30 mM, wherein the composition is suitable for injecting into a diabetic patient to treat diabetes, and
   wherein the composition does not contain any one of
EDTA,
anionic compounds whose chemical structure includes at least one carboxyl functional group to which is covalently bonded an amino-acid residue,
saccharides whose chemical structure includes at least one carboxyl functional group to which is covalently bonded an amino-acid residue, and
oligosaccharides whose chemical structure includes at least one carboxyl functional group to which is covalently bonded an amino-acid residue.

In embodiments, the present disclosure provides an aqueous pharmaceutical composition comprising:
insulin in hexameric form, and citric acid or a salt thereof at a concentration from 6 to 30 mM, wherein the composition is suitable for injecting into a diabetic patient to treat diabetes, and
   wherein the composition does not contain any one of
EDTA,
anionic compounds whose chemical structure includes at least one carboxyl functional group to which is covalently bonded an aromatic amino-acid residue,
saccharides whose chemical structure includes at least one carboxyl functional group to which is covalently bonded an aromatic amino-acid residue, and
oligosaccharides whose chemical structure includes at least one carboxyl functional group to which is covalently bonded an aromatic amino-acid residue.

By “an insulin accelerant other than citrate” is meant a compound, other than citric acid or a salt thereof, which accelerates insulin absorption in the absence of other compounds.

In embodiments, the insulin accelerant other than citric acid or a salt thereof is accelerating the insulin absorption by at least 10% compared with the same composition (same insulin concentration) without said insulin accelerant other than citric acid or a salt thereof.

The acceleration of the insulin absorption is measured with the T50% Cmax insulin.

Examples of insulin accelerants other than citric acid or a salt thereof include hyaluronidase, vasodilators, nicotinic acid and nicotinamide, EDTA, anionic compounds whose chemical structure includes at least one carboxyl functional group to which is covalently bonded an amino-acid residue, saccharides whose chemical structure includes at least one carboxyl functional group to which is covalently bonded an amino-acid residue, and oligosaccharides whose chemical structure includes at least one carboxyl functional group to which is covalently bonded to an amino-acid residue.

In embodiments, the present disclosure provides an aqueous pharmaceutical composition comprising:
insulin in hexameric form, and citric acid or a salt thereof at a concentration from 6 to 30 mM, wherein the composition is suitable for injecting into a diabetic patient to treat diabetes, and
   wherein the composition does not contain more than 80 μg/ml of an insulin accelerant as above defined either alone or in combination with citric acid or a salt thereof.

In embodiments, the composition does not comprise an insulin accelerant other than citric acid or a salt thereof at a concentration above 100, 80, 60 or 40 μg/ml.

In embodiments, the composition comprises an insulin accelerant other than citric acid or a salt thereof at a concentration below 100, 80, 60 or 40 μg/ml.

In embodiments, the composition does not comprise a vasodilator at a concentration above 100, 80, 60 or 40 μg/ml.

In embodiments, the composition comprises a vasodilator at a concentration below 100, 80, 60 or 40 μg/ml.

In embodiments, the vasodilator act by mediating hyperpolarization by blocking calcium ion channels, a cAMP-mediated vasodilatory agent, a cGMP-mediated vasodilatory agent or any combination thereof. In embodiments of the present invention, the vasodilatory agent that can act by mediating hyperpolarization by blocking calcium ion channels is preferably adenosine, endothelium-derived hyperpolarizing factor, a phosphodiesterase type 5 (PDES) inhibitor, a potassium channel opener or any combination thereof.

In embodiments, the vasodilator is chosen from nitroglycerin, a nitric oxide forming agent, amyl nitrite, nitroprusside or any combination thereof.

In embodiments, the composition comprises a concentration of EDTA below 300, 250, 200, 150, 100, 50 or 25 μM.

In embodiments, the composition does not contain EDTA.

In embodiments, the composition is characterized in that it does not contain substituted anionic compounds, polysaccharides, oligosaccharides or substituted citrate as described in US 2014/0187499, US 2014/0142034, US 2013/0231281, US 2016/0015814, US 2016/0082106 or U.S. application Ser. No. 15/353,522, each of which is incorporated by reference herein.

In embodiments, the composition does not contain any species having a saccharide unit or saccharide backbone.

In a particular embodiment, the composition does not contain any of the substituted anionic compounds, substituted citrate or substituted anionic oligosaccharides as defined in the following general formulas I to VI.

In embodiments, the composition does not contain any substituted anionic compound, in isolated form or as a mixture, consisting of a backbone made up of a discrete number u from 1 to 8 (1≤u≤8) of identical or different saccharide units, linked via identical or different glycosidic bonds, said saccharide units being chosen from the group consisting of pentoses, hexoses, uronic acids, N-acetylhexosamines in cyclic form or in open reduced form, characterized in that they are substituted with:
a) at least one substituent of general formula I:

—[R₁]ₐ-[[Q]-[R₂]ₙ]ₘ formula I
the substituents being identical or different when there are at least two substituents, in which:
if n is equal to 0, then the radical -[Q]- is derived from a C₃ to C₁₅ carbon-based chain which is optionally branched or substituted, optionally unsaturated and/or optionally comprising one or more ring(s) and/or comprising at least one heteroatom chosen from O, N and S and at least one function L chosen from amine and alcohol functions, said radical -[Q]- being attached to the backbone of the compound by means of a linker arm R₁ to which it is bonded via a function T, or directly bonded to the backbone via a function G,
if n is equal to 1 or 2, then the radical -[Q]- is derived from a C₂ to C₁₅ carbon-based chain which is optionally branched or substituted, optionally unsaturated and/or optionally comprising one or more ring(s) and/or comprising at least one heteroatom chosen from O, N and S and at least one function L chosen from amine and alcohol functions and bearing n radical(s) R₂, said radical -[Q]- being attached to the backbone of the compound by means of a linker arm R₁ to which it is bonded via a function T, or directly bonded to the backbone via a function G,
the radical —R₁— being:
   either a bond and then a=0, and the radical -[Q]- is directly bonded to the backbone via a function G,
   or a C₂ to C₁₅ carbon-based chain, and then a=1, which is optionally substituted and/or comprising at least one heteroatom chosen from O, N and S and at least one acid function before the reaction with the radical -[Q]-, said chain being bonded to the radical -[Q]- via a function T resulting from the reaction of the acid function of the radical —R₁— with an alcohol or amine function of the precursor of the radical -[Q]-, and said radical R₁ is attached to the backbone by means of a function F resulting from a reaction between a hydroxyl function or a carboxylic acid function borne by the backbone and a function or a substituent borne by the precursor of the radical —R₁—,
the radical —R₂ is a C₁ to C₃₀ carbon-based chain which is optionally branched or substituted, optionally unsaturated and/or optionally comprising one or more ring(s) and/or one or more heteroatom(s) chosen from O, N and S; it forms, with the radical -[Q]-, a function Z resulting from a reaction between the alcohol, amine or acid functions borne by the precursors of the radical —R₂ and of the radical -[Q]-.
F is a function chosen from ether, ester, amide or carbamate functions,
T is a function chosen from amide or ester functions,
Z is a function chosen from ester, carbamate, amide or ether functions,
G is a function chosen from ester, amide or carbamate functions,
n is equal to 0, 1 or 2,
m is equal to 1 or 2,
the degree of substitution of the saccharide units, j, with —[R₁]ₐ-[[AA]-[R₂]ₙ]ₘ being between 0.01 and 6, 0.01≤j≤6;
b) and, optionally, one or more substituents —R′₁,
   the substituent —R′₁ being a C₂ to C₁₅ carbon-based chain which is optionally substituted and/or comprising at least one heteroatom chosen from O, N and S and at least one acid function in the form of an alkali metal cation salt, said chain being bonded to the backbone via a function F′ resulting from a reaction between a hydroxyl function or a carboxylic acid function borne by the backbone and a function or a substituent borne by the precursor of the substituent —R′₁,
the degree of substitution of the saccharide units, i, with —R′₁ being between 0 and 6−j, 0≤i≤6−j and,
if n≠0 and if the backbone does not bear anionic charges before substitution, then i≠0,
—R′₁ identical to or different than —R₁—,
the free salifiable acid functions borne by —R′₁— are in the form of alkali metal cation salts,
F′ is a function chosen from ether, ester, amide or carbamate functions,
F, F′, T, Z and G being identical or different,
i+j≤6, being said that all the above definitions are only given regarding formula I.

In embodiments, the composition does not contain any substituted anionic compound, in isolated form or as a mixture, consisting of a backbone formed from a discrete number u from 1 to 8 (1≤u≤8) of identical or different saccharide units, linked via identical or different glycoside bonds, said saccharide units being chosen from the group consisting of hexoses, in cyclic form or in open reduced form, characterized in that they are substituted with:
a) at least one substituent of general formula II:

—[R₁]ₐ-[AA]ₘ Formula II
the substituents being identical or different when there are at least two substituents, in which:
the radical -[AA] denotes an amino acid residue,
the radical —R₁— being:
   either a bond and then a=0 and the amino acid residue -[AA] is directly linked to the backbone via a function G,
   or a C2 to C15 carbon-based chain, and then a=1, optionally substituted and/or comprising at least one heteroatom chosen from O, N and S and at least one acid function before the reaction with the amino acid, said chain forming with the amino acid residue -[AA] an amide function, and is attached to the backbone by means of a function F resulting from a reaction between a hydroxyl function borne by the backbone and a function or substituent borne by the precursor of the radical —R₁—,
F is a function chosen from ether, ester and carbamate functions,
G is a carbamate function,
m is equal to 1 or 2,
the degree of substitution of the saccharide units, j, in —[R1]ₐ-[AA]ₘ being strictly greater than 0 and less than or equal to 6, 0<j≤6
b) and, optionally, one or more substituents —R′₁,
the substituent —R′₁ being a C2 to C15 carbon-based chain, which is optionally substituted and/or comprising at least one heteroatom chosen from O, N and S and at least one acid function in the form of an alkali metal cation salt, said chain being linked to the backbone via a function F′ resulting from a reaction between a hydroxyl function borne by the backbone and a function or substituent borne by the precursor of the substituent —R′₁,
F′ is an ether, ester or carbamate function,
the degree of substitution of the saccharide units, i, in —R′₁, being between 0 and 6−j, 0≤i≤6−j, and
F and F′ are identical or different,
F and G are identical or different,
i+j≤6,
—R′₁ is identical to or different from —R₁—,
the free salifiable acid functions borne by the substituent —R′₁ are in the form of alkali metal cation salts,
   said glycoside bonds, which may be identical or different, being chosen from the group consisting of glycoside bonds of (1,1), (1,2), (1,3), (1,4) or (1,6) type, in an alpha or beta geometry, being said that all the above definitions are only given regarding formula II.

In embodiments, the composition does not contain any oligosaccharide whose average degree of polymerization is from 3 to 13 and whose polydispersity index PDI is above 1.0, of the following general formula III:

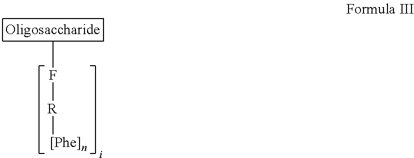

the oligosaccharide is a dextran,
F results from coupling between the linker arm R and an —OH function of the oligosaccharide and being either an ester, carbamate or ether function,
R is a chain comprising between 1 and 15 carbons, optionally branched and/or unsaturated, comprising one or more heteroatoms, such as O, N and/or S, and having at least one carboxyl function,
Phe is a residue of a phenylalanine derivative, of absolute configuration L or D, produced from coupling between the amine function of the phenylalanine derivative and at least one acid function carried by group R prior to attachment to Phe,
n represents the mole fraction of the R substituted with Phe and is between 0.1 and 0.9, preferably between 0.2 and 0.8, more preferably between 0.3 and 0.7, more preferably between 0.3 and 0.5;
i represents the average mole fraction of the groups F—R-[Phe]n borne per saccharide unit and is between 0.5 and 3.0, preferably between 1.0 and 2.5, preferably between 1.2 and 2.2, preferably between 1.4 and 2.0;
when R is not substituted with Phe, the acid or acids of group R are carboxylates with an alkaline cation, preferably such as Na⁺, K⁺, Ca²⁺ or Mg²⁺, being said that all the above definitions are only given regarding formula III

In embodiments, the composition does not contain any substituted anionic compound of formula IV, said formula IV representing the saccharide unit in open form in which at most one from among R₂, R₃, R₄ and R₆ represents a saccharide backbone formed from a discrete number of closed saccharide units:

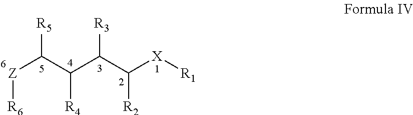

in which
1) Z is either a radical —C═O—, or a radical —CH₂—,
2) X is either a radical —C═O—, or a radical —CH₂—,
3) R₅ is either an —OH radical, or a radical -f-[A]-COOH,
4) R₂, R₃, R₄, R₆, which may be identical or different, are chosen from the group consisting of the radicals —OH, -f-[A]-COOH and at most one from among R₂, R₃, R₄, R₆ is a radical resulting from a saccharide backbone formed from a discrete number n−1 of between 1 and 7 (1≤n−1≤7) of identical or different closed saccharide units, the hydroxyl functions of which may or may not be substituted with a radical -f-[A]-COOH,
5) -[A]- is an at least divalent radical comprising from 1 to 4 carbon atoms chosen from the group consisting of alkyl radicals —(CH₂)ₓ— 1≤x≤4, radicals comprising at least one heteroatom chosen from O, N and S and radicals bearing carboxyl functions and/or -f-[A]-COOH is resulting from an amino acid or an acid alcohol, comprising from 2 to 5 carbon atoms, and is linked to the saccharide units of the compound via a function f;
6) f is chosen from the group consisting of ether, carbamate and amide functions;
7) R₁ is a radical —N(L)ₛ([E]-(o-[AA])ᵤ)ₜ or —N(L)ₛ[AA]
   the linker arm -E- is an at least divalent radical comprising from 1 to 10 carbon atoms optionally comprising at least one heteroatom chosen from O, N and S, and optionally bearing carboxyl functions, and/or —N(L)ₛ-([E]-(o-)ᵤ) is resulting from an amino acid, a diamine, an amino alcohol, an amino diacid, a triamine, a tetramine, an amino diol or an amino triol comprising from 2 to 12 carbon atoms, the amine functions of which are primary and/or secondary;
   -[AA] is resulting from an aromatic amino acid comprising a phenyl or an indole, which may or may not be substituted, or an aromatic amino acid derivative containing a phenyl or an indole, which may or may not be substituted,
   o is an amide, carbamate or carbamide function,
   u=1, 2 or 3 and
   when R₁ is a radical —N(L)ₛ([E]-(o-[AA])ᵤ)ₜ and
      when X is a radical —C═O— then
         s=0 or 1, t=1 or 2 and s+t=2;
         L is chosen from the group consisting of
         H, and
         a linear or branched alkyl radical comprising from 1 to 4 carbon atoms, and
      when X is a radical —CH₂—, then
         s=0, 1 or 2, t=1 or 2 and s+t=2 or 3;
         if s=1, L is chosen from the group consisting of
         —H, and
         —H and/or -[A]-COOH if f as defined in point 6) is an ether function,
         —H and/or —CO—NH-[A]-COOH if f as defined in point 6) is a carbamate function, and
         a linear or branched alkyl radical comprising from 1 to 4 carbon atoms, and
         if s=2, L is chosen from the group consisting of:
         —H, and
         —H and/or -[A]-COOH if f as defined in point 6) is an ether function, and
         a linear or branched alkyl radical comprising from 1 to 4 carbon atoms, and
   when R₁ is a radical —N(L)ₛ[AA] and
      when X is a radical —C═O— then s=1 and L is chosen from the group consisting of:
         —H,
         a linear or branched alkyl radical comprising from 1 to 4 carbon atoms,
      when X is a radical —CH₂—, then s=1 or 2 and
         if s=1, L is chosen from the group consisting of:
         —H, and
         —H and/or -[A]-COOH if f as defined in point 6) is an ether function,
         —H and/or —CO—NH-[A]-COOH if f as defined in point 6) is a carbamate function, and
         a linear or branched alkyl radical comprising from 1 to 4 carbon atoms, and
         if s=2, L is chosen from the group consisting of
         —H, and
         —H and/or -[A]-COOH if f as defined in point 6) is an ether function, and
         a linear or branched alkyl radical comprising from 1 to 4 carbon atoms, and
8) the degree of substitution represented by p is the number of carboxylate functions per saccharide unit, said carboxylate functions optionally being carboxylate functions that are naturally present on the saccharide units, being resulting from substitution with radicals -[A]-COOH and/or radicals -[AA] and 6≥p≥0.1,
   and the acid functions being in the form of salts of alkali metal cations chosen from the group consisting of Na⁺ and K⁺ being said that all the above definitions are only given regarding formula IV.

In embodiments, the composition does not contain any substituted anionic compound corresponding to formula V below:

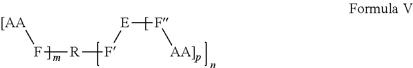

wherein
R represents a saturated or unsaturated, linear, branched or cyclic hydrocarbon-based radical comprising from 1 to 12 carbon atoms, optionally comprising at least one function chosen from ether, alcohol and carboxylic acid functions.
AA is a radical resulting from an aromatic amino acid comprising a phenyl group or an indole group, which is substituted or not substituted, or an aromatic amino acid derivative comprising a phenyl group or an indole group, which is substituted or not substituted, said radical AA bearing at least one free acid function,
E represents an at least divalent radical, comprising from 2 to 6 carbon atoms,
F, F′ and F″ represent, independently of each other, a function chosen from amide, carbamate and urea functions, F and F″ being functions resulting from a reaction involving the amine of the aromatic amino acid, the precursor of the radical AA, F′ being a function involving a reactive function of the precursor of R and a reactive function of the precursor of E,
p being an integer between 1 and 3,
m is an integer between 0 and 6; n is an integer between 0 and 6; m+n is an integer between 1 and 6;
said compound comprising at least two carboxylic acid functions in the form of a salt of an alkali metal chosen from Na⁺ and K⁺, being said that all the above definitions are only given regarding formula V

In embodiments, the composition does not contain any substituted citrate of formula VI:

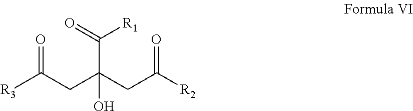

in which:
R₁, R₂, R₃, identical or different, represent OH or AA,
at least one of the R₁, R₂, R₃ is an AA radical,
AA is a radical resulting from a natural or synthetic aromatic amino acid comprising at least one phenyl group or indole group, substituted or not substituted, said AA radical having at least one free carboxylic acid function,
the carboxylic acid functions are in the form of a salt of an alkali metal selected from Na⁺ and K⁺, being said that all the above definitions are only given regarding formula VI

In embodiments, the pH of the composition is from 6 to 8, such as from 6.8 to 7.8, such as from 7.0 to 7.8. In some embodiments, the pH of the composition is 7.0, 7.1, 7.2, 7.3, 7.4, 7.5, 7.6, 7.7, or 7.8+/−0.1 pH units.

The units recommended for the insulins by the pharmacopoeias are presented in the table below with their equivalents in mg:

| | Pharmacopoeia EP 8.0 | Pharmacopoeia US - USP38 |
|---|---|---|
| Insulin | (2014) | (2015) |
| Aspart | 1 U = 0.0350 mg of insulin | 1 USP = 0.0350 mg of insulin |
| | aspart | aspart |
| Lispro | 1 U = 0.0347 mg of insulin | 1 USP = 0.0347 mg of insulin |
| | lispro | lispro |
| Human | 1 IU = 0.0347 mg of human | 1 USP = 0.0347 mg of human |
| | insulin | insulin |

Nevertheless, in the continuation of the text, IU is used systematically for the quantities and the concentrations, interchangeably, of all the insulins. The respective equivalent values in mg are those given above for the values expressed in U, IU or USP.

In embodiments, the citric acid salt is an alkali metal salt selected from Na⁺ and K⁺.

In embodiments, the composition is characterized in that the concentration of citric acid or a salt thereof is from 2 to 500 mM.

In embodiments, the composition is characterized in that the concentration of citric acid or a salt thereof is from 2 to 250 mM.

In embodiments, the composition is characterized in that the concentration of citric acid or a salt thereof is from 2 to 150 mM.

In embodiments, the composition is characterized in that the concentration of citric acid or a salt thereof is from 2 to 120 mM.

In embodiments, the composition is characterized in that the concentration of citric acid or a salt thereof is from 2 to 100 mM.

In embodiments, the composition is characterized in that the concentration of citric acid or a salt thereof is from 2 to 65 mM.

In embodiments, the composition is characterized in that the concentration of citric acid or a salt thereof is from 2 to 50 mM.

In embodiments, the composition is characterized in that the concentration of citric acid or a salt thereof is from 2 to 30 mM.

In embodiments, the composition is characterized in that the concentration of citric acid or a salt thereof is from 2 to 20 mM.

In embodiments, the composition is characterized in that the concentration of citric acid or a salt thereof is from 6 to 500 mM.

In embodiments, the composition is characterized in that the concentration of citric acid or a salt thereof is from 6 to 250 mM.

In embodiments, the composition is characterized in that the concentration of citric acid or a salt thereof is from 6 to 150 mM.

In embodiments, the composition is characterized in that the concentration of citric acid or a salt thereof is from 6 to 120 mM.

In embodiments, the composition is characterized in that the concentration of citric acid or a salt thereof is from 6 to 50 mM.

In embodiments, the composition is characterized in that the concentration of citric acid or a salt thereof is from 6 to 30 Mm.

In embodiments, the composition is characterized in that the concentration of citric acid or a salt thereof is from 7 to 100 mM.

In embodiments, the composition is characterized in that the concentration of citric acid or a salt thereof is from 7 to 65 mM.

In embodiments, the composition is characterized in that the concentration of citric acid or a salt thereof is from 7 to 50 mM.

In embodiments, the composition is characterized in that the concentration of citric acid or a salt thereof is from 7 to 30 mM

In embodiments, the composition is characterized in that the concentration of citric acid or a salt thereof is from 8 to 50 mM.

In embodiments, the composition is characterized in that the concentration of citric acid or a salt thereof is from 8 to 30 mM.

In embodiments, the composition is characterized in that the concentration of citric acid or a salt thereof is from 9 to 30 mM.

In embodiments, the composition is characterized in that the concentration of citric acid or a salt thereof is from 9 to 20 mM.

In embodiments, the composition is characterized in that the concentration of citric acid or a salt thereof is from 9.3 to 18.6 mM.

In embodiments, the composition is characterized in that the concentration of citric acid or a salt thereof is 9.3 mM.

In embodiments, the composition is characterized in that the concentration of citric acid or a salt thereof is 18.6 mM.

In embodiments, the composition is characterized in that the concentration of citric acid or a salt thereof is 27.9 mM.

In embodiments, the composition is characterized in that the concentration of citric acid or a salt thereof is 37.2 mM.

In embodiments, the composition is characterized in that the concentration of citric acid or a salt thereof is 46.5 mM.

In some embodiments, the pharmaceutical composition comprises a therapeutically effective dose of insulin. For instance, in embodiments, the pharmaceutical composition is characterized in that the concentration of insulin is from 240 to 3000 μM (40 to 500 IU/ml).

In embodiments, the composition is characterized in that the pharmaceutical composition is characterized in that the concentration of insulin is from 600 to 3000 μM (100 to 500 IU/ml).

In embodiments, the composition is characterized in that the pharmaceutical composition is characterized in that the concentration of insulin is from 600 to 2400 μM (100 to 400 IU/ml).

In embodiments, the composition is characterized in that the pharmaceutical composition is characterized in that the concentration of insulin is from 600 to 1800 μM (100 to 300 IU/ml).

In embodiments, the composition is characterized in that the pharmaceutical composition is characterized in that the concentration of insulin is from 600 to 1200 μM (100 to 200 IU/ml).

In embodiments, the composition is characterized in that it relates to a pharmaceutical composition characterized in that the concentration of insulin is 600 μM (100 IU/ml), 1200 μM (200 IU/ml), 1800 μM (300 IU/ml), 2400 μM (400 IU/ml) or 3000 μM (500 IU/ml).

In embodiments, the composition is characterized in that the molar ratios of citric acid or salt thereof/insulin are from 3 to 800.

In embodiments, the composition is characterized in that the molar ratios of citric acid or salt thereof/insulin are from 3 to 400.

In embodiments, the composition is characterized in that the molar ratios of citric acid or salt thereof/insulin are from 3 to 250.

In embodiments, the composition is characterized in that the molar ratios of citric acid or salt thereof/insulin salt are from 3 to 200.

In embodiments, the composition is characterized in that the molar ratios of citric acid or salt thereof/insulin salt are from 3 to 160.

In embodiments, the composition is characterized in that the molar ratios of citric acid or salt thereof/insulin salt are from 3 to 110.

In embodiments, the composition is characterized in that the molar ratios of citric acid or salt thereof/insulin salt are from 3 to 80.

In embodiments, the composition is characterized in that the molar ratios of citric acid or salt thereof/insulin salt are from 3 to 50.

In embodiments, the composition is characterized in that the molar ratios of citric acid or salt thereof/insulin salt are from 3 to 35.

In embodiments, the composition is characterized in that the molar ratios of citric acid or salt thereof/insulin salt are from 10 to 250.

In embodiments, the composition is characterized in that the molar ratios of citric acid or salt thereof/insulin salt are from 10 to 200.

In embodiments, the composition is characterized in that the molar ratios of citric acid or salt thereof/insulin salt are from 11 to 160.

In embodiments, the composition is characterized in that the molar ratios of citric acid or salt thereof/insulin salt are from 11 to 100.

In embodiments, the composition is characterized in that the molar ratios of citric acid or salt thereof/insulin salt are from 13 to 85.

In embodiments, the composition is characterized in that the molar ratios of citric acid or salt thereof/insulin salt are from 13 to 50.

In embodiments, the composition is characterized in that the molar ratios of citric acid or salt thereof/insulin salt are from 15 to 35.

In embodiments, the composition is characterized in that the molar ratios of citric acid or salt thereof/insulin salt are from 15 to 32.

In embodiments, the composition is characterized in that the molar ratio of citric acid or salt thereof to insulin is equal to 15, 23, 32, and 46.

In the molar ratios above, the number of moles of insulin is meant as the number of moles of insulin monomer.

In embodiments, the composition is characterized in that the concentration of citric acid or salt thereof is comprised from 2 to 129 mg per 100 IU of insulin.

In embodiments, the composition is characterized in that the concentration of citric acid or salt thereof is comprised from 2 to 35 mg per 100 IU of insulin.

In embodiments, the composition is characterized in that the concentration of citric acid or salt thereof is comprised from 2 to 13 mg per 100 IU of insulin.

In embodiments, the composition is characterized in that the concentration of citric acid or salt thereof is comprised from 2 to 8 mg per 100 IU of insulin.

In embodiments, the composition is characterized in that the concentration of citric acid or salt thereof is comprised from 2.3 to 5.2 mg per 100 IU of insulin.

In embodiments, the composition is characterized in that the concentration of citric acid or salt thereof is 2.4 mg per 100 IU of insulin.

In embodiments, the composition is characterized in that the concentration of citric acid or salt thereof is 4.8 mg per 100 IU of insulin.

In embodiments, the composition is characterized in that the concentration of citric acid or salt thereof is 7.2 mg per 100 IU of insulin.

It is known to those skilled in the art that the onset of action of insulins is dependent on the insulin concentration. Only the onset time values of the compositions at 100 IU/ml are documented.

The disclosure also relates to a method for preparing an insulin composition having an insulin concentration comprised from 240 to 3000 μM (40 to 500 IU/ml), whose onset of action and/or of appearance in humans is more rapid than that of the reference composition at the same concentration of insulin in the absence of citric acid or a salt thereof, characterized in that it comprises a step of adding to said composition at least one citric acid or a salt thereof.

In embodiments of the method, the insulin is in hexameric form.

The disclosure also relates to a method for preparing an insulin composition having an insulin concentration comprised from 600 to 1200 μM (100 to 200 IU/ml), whose onset of action and/or of appearance in humans is more rapid than that of the reference composition at the same concentration of insulin in the absence of a citric acid or a salt thereof, characterized in that it comprises a step of adding to said composition at least one citric acid or a salt thereof.

In embodiments, the disclosure also relates to a method for preparing an insulin composition having an insulin concentration comprised from 600 to 3000 μM (100 to 500 IU/ml) such as 600 μM (100 IU/ml), 1200 μM (200 IU/ml), 1800 μM (300 IU/ml) 2400 μM (400 IU/ml) or 3000 μM (500 IU/ml) whose onset of action and/or of appearance in humans is at least 5%, 10%, 15%, etc. more rapid than that of the reference composition at the same concentration of insulin in the absence of citric acid or a salt thereof. In embodiments, the disclosure relates to a method for preparing a composition, in the form of an aqueous solution, comprising insulin in hexameric form and at least one citric acid or a salt thereof for use in a method for treating diabetic patients, characterized in that it allows improving the rapid absorption of insulin.

In particular, it allows increasing the area under the curve of the insulin concentration in the serum as a function of time (AUC) between 0 and 15 minutes or 0 and 30 minutes after administration (AUC-Ins0-15 or 0-30 min) by at least 10%, 15%, 20%, 25%, 30%, 40%, 50%, 60%, 70%, 80%, or 90%. relative to the reference composition at the same concentration of insulin in the absence of citric acid or a salt thereof.

In embodiments, the disclosure relates to a method for preparing a composition, in the form of an aqueous solution, comprising insulin in hexameric form and at least one citric acid or a salt thereof for use in a method for treating diabetic patients, characterized in that it allows improving the rapid absorption of insulin, said improvement being measured by the increase of at least 10% of the area under the insulin concentration curve in serum as a function of time (AUC) between 0 and 15 minutes or 0 and 30 minutes after administration (AUC-Ins0-15 or 0-30 min), with respect to the reference composition at the same concentration of insulin in the absence of citric acid or a salt thereof.

In embodiments, the disclosure relates to a method for preparing a composition, in the form of an aqueous solution, comprising insulin in hexameric form and at least one citric acid or a salt thereof for use in a method for treating diabetic patients, characterized in that it allows improving the rapid absorption of insulin, said improvement being measured by the increase of at least 30% of the area under the insulin concentration curve in serum as a function of time (AUC) between 0 and 15 minutes or 0 and 30 minutes after administration (AUC-Ins0-15 or 0-30 min), with respect to the reference composition at the same concentration of insulin in the absence of citric acid or a salt thereof.

In embodiments, the disclosure relates to a method for preparing a composition, in the form of an aqueous solution, comprising insulin in hexameric form and at least one citric acid or a salt thereof for use in a method for treating diabetic patients, characterized in that it allows improving the rapid absorption of insulin, said improvement being measured by the increase of at least 50% of the area under the insulin concentration curve in serum as a function of time (AUC) between 0 and 15 minutes or 0 and 30 minutes after administration (AUC-Ins0-15 or 0-30 min), with respect to the reference composition at the same concentration of insulin in the absence of citric acid or a salt thereof.

In embodiments, the disclosure relates to a method for preparing a composition, in the form of an aqueous solution, comprising insulin in hexameric form and at least one citric acid or a salt thereof for use in a method for treating diabetic patients, characterized in that it allows improving the rapid absorption of insulin, said improvement being measured by the increase of at least 70% of the area under the insulin concentration curve in serum as a function of time (AUC) between 0 and 15 minutes or 0 and 30 minutes after administration (AUC-Ins0-15 or 0-30 min), with respect to the reference composition at the same concentration of insulin in the absence of citric acid or a salt thereof.

In embodiments, the disclosure relates to a method for preparing a composition, in the form of an aqueous solution, comprising insulin in hexameric form and at least one citric acid or a salt thereof for use in a method for treating diabetic patients, characterized in that it allows improving the rapid absorption of insulin, said improvement being measured by the increase of at least 90% of the area under the insulin concentration curve in serum as a function of time (AUC) between 0 and 15 minutes or 0 and 30 minutes after administration (AUC-Ins0-15 or 30 min), with respect to the reference composition at the same concentration of insulin in the absence of citric acid or a salt thereof.

In embodiments, the disclosure relates to the preparation of an insulin composition called ultrafast, characterized in that it comprises a step of adding to said composition at least one citric acid or a salt thereof.

In embodiments, the insulin is in hexameric form.

In embodiments, the pH of the composition is comprised from 6 to 8.

In embodiments of the method, the pH of the composition is comprised from 6.8 to 7.8.

In embodiments of the method, the pH of the composition is comprised from 7.0 to 7.8.

In embodiments, the disclosure relates to the use of at least one citric acid salt, for preparing an insulin composition, allowing, after administration, to accelerate the passage of insulin into the bloodstream and to reduce glycemia more rapidly compared to a composition free of citric acid or a salt thereof.

In embodiments of the use, the pH of the composition is comprised from 6 to 8.

In embodiments, the citric acid salt is an alkali metal salt selected from Na⁺ and K+.

The disclosure also relates to a pharmaceutical composition characterized in that it is obtained by drying to/or lyophilization.

In embodiments, the compositions according also comprise the addition of zinc salts having at a concentration comprised from 0 to 500 μM, in particular from 0 to 300 μM, and more particularly from 0 to 200 μM.

In embodiments, the compositions comprise buffers at concentrations comprised from 0 to 100 mM, preferably from 0 to 50 mM, and even from 15 to 50 mM.

In embodiments, the buffer is Tris.

In embodiments, the buffer is a phosphate salt.

In embodiments, the phosphate salt is selected from the group consisting of sodium dihydrogenophosphate, sodium hydrogenophosphate and sodium phosphate.

In embodiments, the compositions also comprise preservatives.

In embodiments, the preservatives are selected from the group consisting of m-cresol and phenol, alone or as a mixture.

In embodiments, the concentration of the preservatives is comprised from 10 to 50 mM, in particular from 10 to 40 mM.

In embodiments, the compositions may further comprise additives such as tonicity agents such as glycerin, sodium chloride (NaCl), mannitol and glycine.

In embodiments, the concentration of the glycerin is comprised from 100 to 250 mM.

In embodiments, the concentration of the glycerin is comprised from 125 to 225 mM.

In embodiments, the concentration of the glycerin is comprised from 150 to 200 mM.

In embodiments, the concentration of the sodium chloride is comprised from 1 to 60 mM.

In embodiments, the concentration of the sodium chloride is comprised from 5 to 25 mM.

In embodiments, the concentration of the sodium chloride is 10 mM.

In embodiments, the compositions may also comprise additives in accordance with pharmacopoeias such as surfactants.

In embodiments the surfactants are non-ionic surfactants.

In embodiments the surfactants are polysorbate or poloxamer.

In embodiments, the polysorbate is chosen from the group comprising Polysorbate 80 (Tween 80) which is derived from polyethoxylated sorbitan and oleic acid and Polysorbate 20 (Tween 20) which is derived from polyethoxylated sorbitan and lauric acid.

In embodiments, the surfactant can have a stabilizing effect on the composition. In some cases, the addition of citric acid or a salt thereof to an insulin composition can induce a physical destabilization of the composition.

In embodiments, the concentration of surfactant in the composition is from 0.0002 to 0.2% w/v.

In embodiments, the concentration of surfactant is comprised from 0.0005 to 0.2% w/v.

In embodiments, the concentration of surfactant is comprised from 0.002 to 0.2% w/v.

In embodiments, the compositions also comprise a magnesium and/or calcium salt.

In embodiments, the compositions also comprise a magnesium salt Magnesium salt may help to limit irritation at the injection site and/or enhance the stability of the composition.

In embodiments, the magnesium salt is selected from the group comprising magnesium hydroxide, magnesium sulfate, magnesium sulfate heptahydrate, magnesium pyrophosphate, magnesium oxide and magnesium halides such as magnesium chloride, magnesium bromide or magnesium iodide.

In embodiments, the concentration of magnesium (Mg²⁺) or calcium salt (Ca²⁺) is comprised from 0.4 to 10 mM.

In embodiments, the concentration of magnesium (Mg²⁺) is comprised from 0.4 to 10 mM.

Solutions of HCl (such as 10%) or NaOH (such as 10%) may also be added to the compositions in order to adjust the pH.

In embodiments, total chloride concentration is comprised from 1 to 60 mM.

In embodiments, total chloride concentration is comprised from 5 to 60 mM.

In embodiments, total chloride concentration is comprised from 10 to 60 mM.

In embodiments, the compositions may also comprise all the excipients in accordance with pharmacopoeias and compatible with the insulins used at the usual concentrations.

In the case of local and systemic releases, the modes of administration considered are intravenous, subcutaneous, intradermal or intramuscular route. In particular, the mode of administration is the subcutaneous route.

Transdermal, oral, nasal, vaginal, ocular, buccal, pulmonary routes of administration are also considered.

In embodiments, the disclosure provides a container comprising a composition such as defined herein.

In embodiments, the container is closed and/or sealed. In embodiments, the container comprises 1 to 20 ml of the composition, such as 1 to 10 or 1 to 5 ml of the composition, including 1, 2, 3, 4, 5, 6, 7, 8, 9, or 10 ml of the composition.

In embodiments, the container comprises 2 to 5 ml of the composition.

In embodiments, the container comprises 3 to 5 ml of the composition.

In embodiments the container is connected with an injection device.

In embodiments the container is a cartridge, a reservoir, a vial or a prefilled syringe. In embodiments, the cartridge is removable from and/or insertable into the injection device.

In embodiments the cartridge is adapted to be connected to a pen, an auto-injector, a syringe or a pump.

In embodiments, the container is a reservoir connected to the injection device.

In embodiments the container is connected to an auto-injector or a syringe.

In embodiments the container or the device to which it is connected is wrapped, for example wrapped with cardboard and/or plastic.

In embodiments the container is a vial. In embodiments, the vial has a volume of 1 to 20 ml, such as 5 to 20 ml, including 2 ml, 3 ml, 4 ml, 5 ml, 10 ml, 15 ml, or 20 ml. In some embodiments, the vile includes a stopper.

In embodiments the container comprises 1 to 20 ml of the composition, such as 5 to 20 ml of the composition, including 2 ml, 3 ml, 4 ml, 5 ml, 10 ml, 15 ml, or 20 ml of the composition.

In embodiments the container comprises 10 ml of the composition.

In embodiments the container comprises a ratio liquid/gas v/v of more than 1000.

In embodiments the injection device comprising the composition is a pen, an autoinjector, a syringe, a pre-filled syringe, or a pump.

In embodiments, the pump is a closed loop pump.

In embodiments, the pump is an open loop pump.

In embodiments, the pump is an injection pump.

In embodiments, the pump is a patch pump.

In embodiments the disclosure provides a kit comprising the composition and instructions explaining how to use it.

In embodiments, the disclosure provides a kit comprising the composition, an injection device and instructions explaining how to use it.

In embodiments, the disclosure provides a pump comprising a container comprising the composition.

In some embodiments, the present disclosure provides for a unit dose or a plurality of unit doses comprising a composition described herein. In some instances, the container described herein, such as a cartridge, contains a unit dose or a plurality of unit doses, such as 1-12 unit doses, such as 1, 2, 3, 4, 5, 6, 7, 8, 9, 10, 11, or 12 unit doses.

### Examples

I. Insulin Lispro

### Part I.A: Preparation of Solutions

### I.A1. Humalog® Fast-Acting Analog Insulin Solution at 100 IU/Ml

This solution is a commercial solution of insulin lispro from Eli Lilly sold under the name of Humalog® U100. This product is a fast-acting analog insulin. In the present text, when the term Humalog® is used without further precision, it refers to Humalog® U100, and when the expression “commercial formulation of insulin lispro” is used without further precision, it refers to the commercial formulation of insulin lispro at 100 IU/ml. Humalog® U100 has the following composition: glycerin (16 mg/ml), dibasic sodium phosphate (1.88 mg/ml), meta-cresol (3.15 mg/ml), zinc oxide (content adjusted to provide 19.7 μg/ml zinc ion), water for injection. HCl 10% and/or and NaOH 10% may be added to adjust pH. Insulin lispro has a pH of 7.0 to 7.8 (Eli Lilly and Company, Humalog® FDA label, AAD_0025 NL 5532 AMP—https://www.accessdata.fda.gov/drugsatfda_docs/label/2007/020563s075,021017s040,021018s034lbl.pdf).

### I.A2. Preparation of an Insulin Lispro Solution at 200 IU/Ml

The commercial formulation of insulin lispro (Humalog®) at 100 IU/ml was concentrated by using Amicon Ultra-15 centrifuge tubes with a 3 kDa cut-off. The Amicon tubes were first rinsed with 12 ml of deionized water. 12 ml of the commercial formulation were centrifuged for 35 minutes at 4000 g at 20° C. The volume of the retentate was measured and the concentration thus estimated. All retentates were pooled and the overall concentration was estimated (>200 IU/ml).

The concentration of this concentrated insulin lispro solution was adjusted to 200 IU/ml by the addition of the commercial formulation of insulin lispro (Humalog®). The concentrated formulation of insulin lispro has the same concentrations of excipients (m-cresol, glycerin, phosphate) as the commercial formulation at 100 IU/ml.

The final pH was adjusted to 7.4±0.4. The clear solution was filtered through a 0.22 μm membrane and stored at 4° C.

### I.A3. Preparation of an Insulin Lispro Solution at 300, 400 and 500 IU/Ml

Concentrated formulations of insulin lispro at 300 IU/ml, 400 IU/ml or 500 IU/ml (and at all intermediate concentrations) were prepared on the basis of the protocol of Example I.A2 relating to the preparation of an insulin lispro solution at 200 IU/ml. The commercial formulation of insulin was concentrated using Amicon Ultra-15 centrifuge tubes with a 3 kDa cut-off. The Amicon tubes were first rinsed with 12 ml of deionized water. 12 ml of the commercial formulation were centrifuged at 4000 g and 20° C. By controlling the centrifugation time, it was possible to adjust the final insulin concentration in the formulation. The volume of the retentate was measured and the concentration thus estimated. All retentates were pooled and the overall concentration was estimated (>300, 400 or 500 IU/ml).

The concentration of this concentrated insulin solution was adjusted to the desired concentration (e.g. 300 IU/ml, 400 IU/ml or 500 IU/ml) by adding the commercial formulation of insulin lispro (Humalog®). The concentrated insulin formulation has the same excipient concentrations as the commercial formulation at 100 IU/ml.

The final pH was adjusted to 7.4±0.4. The clear solution was filtered through a 0.22 μm membrane and stored at 4° C.

### I.A4. Preparation of an Insulin Lispro Solution at 100 IU/Ml in the Presence of 9.3 mM Citric Acid Salt

For a final volume of 100 ml of composition with a concentration of 9.3 mM citric acid salt, the various reagents were added up in the amounts specified below and in the following order:

| | | |
|---|---|---|
| Sodium citrate | 240 | mg |
| Commercial solution of Humalog ® | 100 | ml |

The final pH was adjusted to 7.4±0.4. The clear solution was filtered through a 0.22 μm membrane and stored at 4° C.

### I.A5. Preparation of an Insulin Lispro Solution at 200 IU/Ml in the Presence of 18.6 mM Citric Acid Salt

For a final volume of 100 ml of formulation with a concentration of 18.6 mM citric acid salt, the various reagents were added up in the amounts specified below and in the following order:

| | | |
|---|---|---|
| Sodium citrate | 480 | mg |
| Insulin lispro at 200 IU/mL | 100 | ml |

The final pH was adjusted to 7.4±0.4. The clear solution was filtered through a 0.22 μm membrane and stored at 4° C.

### I.A6. Preparation of an Insulin Lispro Solution at 300 IU/Ml in the Presence of 27.9 mM Citric Acid Salt.

For a final volume of 100 ml of formulation with a concentration of 27.9 mM citric acid salt, the various reagents were added up in the amounts specified below and in the following order:

| | | |
|---|---|---|
| Sodium citrate | 720 | mg |
| Insulin lispro at 300 IU/mL | 100 | ml |

The final pH was adjusted to 7.4±0.4. The clear solution was filtered through a 0.22 μm membrane and stored at 4° C.

### I.A7. Preparation of an Insulin Lispro Solution at 400 IU/Ml in the Presence of 37.2 mM Citric Acid Salt.

For a final volume of 100 ml of formulation with a concentration of 37.2 mM citric acid salt, the various reagents were added up in the amounts specified below and in the following order:

| | | |
|---|---|---|
| Sodium citrate | 960 | mg |
| Insulin lispro at 400 IU/mL | 100 | ml |

The final pH was adjusted to 7.4±0.4. The clear solution was filtered through a 0.22 μm membrane and stored at 4° C.

### I.A8. Preparation of an Insulin Lispro Solution at 500 IU/Ml in the Presence of 46.5 mM Citric Acid Salt

For a final volume of 100 ml of formulation with a concentration of 46.5 mM citric acid salt, the various reagents were added up in the amounts specified below and in the following order:

| | | |
|---|---|---|
| Sodium citrate | 1200 | mg |
| Insulin lispro at 500 IU/mL | 100 | ml |

The final pH was adjusted to 7.4±0.4. The clear solution was filtered through a 0.22 μm membrane and stored at 4° C.

### I.A9. Preparation of an Insulin Lispro Solution at 100 IU/Ml in the Presence of 18.6 mM Citric Acid Salt

For a final volume of 100 ml of formulation with a concentration of 18.6 mM citric acid salt, the various reagents were added up in the amounts specified below and in the following order:

| | | |
|---|---|---|
| Sodium citrate | 480 | mg |
| Commercial solution of Humalog ® | 100 | ml |

The final pH was adjusted to 7.4±0.4. The clear solution was filtered through a 0.22 μm membrane and stored at 4° C.

### I.A10. Preparation of an Insulin Lispro Solution at 200 IU/Ml in the Presence of 37.2 mM Citric Acid Salt

For a final volume of 100 ml of formulation with a concentration of 37.2 mM citric acid salt, the various reagents were added up in the amounts specified below and in the following order:

| | | |
|---|---|---|
| Sodium citrate | 960 | mg |
| Insulin lispro at 200 IU/mL | 100 | ml |

The final pH was adjusted to 7.4±0.4. The clear solution was filtered through a 0.22 μm membrane and stored at 4° C.

### I.A11. Preparation of an Insulin Lispro Solution at 300 IU/Ml in the Presence of 55.8 mM Citric Acid Salt

For a final volume of 100 ml of formulation with a concentration of 55.8 mM citric acid salt, the various reagents were added up in the amounts specified below and in the following order:

| | | |
|---|---|---|
| Sodium citrate | 1440 | mg |
| Insulin lispro at 300 IU/mL | 100 | ml |

The final pH was adjusted to 7.4±0.4. The clear solution was filtered through a 0.22 μm membrane and stored at 4° C.

### I.A12. Preparation of an Insulin Lispro Solution at 400 IU/Ml in the Presence of 74.4 mM Citric Acid Salt

For a final volume of 100 ml of formulation with a concentration of 74.4 mM citric acid salt, the various reagents were added up in the amounts specified below and in the following order:

| | | |
|---|---|---|
| Sodium citrate | 1920 | mg |
| Insulin lispro at 400 IU/mL | 100 | ml |

The final pH was adjusted to 7.4±0.4. The clear solution was filtered through a 0.22 μm membrane and stored at 4° C.

### I.A13. Preparation of an Insulin Lispro Solution at 500 IU/Ml in the Presence of 93.0 mM Citric Acid Salt

For a final volume of 100 ml of formulation with a concentration of 93.0 mM citric acid salt, the various reagents were added up in the amounts specified below and in the following order:

| | | |
|---|---|---|
| Sodium citrate | 2400 | mg |
| Insulin lispro at 500 IU/mL | 100 | ml |

The final pH was adjusted to 7.4±0.4. The clear solution was filtered through a 0.22 μm membrane and stored at 4° C.

### I.A14. Preparation of an Insulin Lispro Solution at 100 IU/Ml in the Presence of EDTA at 300 μM

For a final volume of 100 ml of formulation with a concentration of 300 μM EDTA, the various reagents were added up in the amounts specified below and in the following order:

| | | |
|---|---|---|
| Commercial solution of Humalog ® at 100 IU/ml | 100 | ml |
| Commercial solution of EDTA at 0.5M | 60 | μL |

The final pH was adjusted to 7.4±0.4. The clear solution was filtered through a 0.22 μm membrane and stored at 4° C.

### I.A15. Preparation of an Insulin Lispro Solution at 100 IU/Ml in the Presence of 4.6 mM Citric Acid Salt

For a final volume of 7.8 ml of formulation with a concentration of 4.6 mM citric acid sodium salt, the various reagents were added up in the amounts specified below and in the following order:

| | | |
|---|---|---|
| Solution of sodium citrate at 0.948 mM | 37.8 | μl |
| Commercial solution of Humalog ® | 7.769 | ml |

The final pH was adjusted to 7.4±0.4. The clear solution was filtered through a 0.22 μm membrane and stored at 4° C.

### I.A16. Preparation of an Insulin Lispro Solution at 100 IU/Ml in the Presence of 4.6 mM Citric Acid Salt and 4 μM Polysorbate 20 (or PS20)

For a final volume of 7.8 ml of formulation with a concentration of 4.6 mM citric acid sodium salt and 4 μM polysorbate 20 (CAS 9005-64-5), the various reagents were added up in the amounts specified below and in the following order:

| | | |
|---|---|---|
| Solution of sodium citrate at 0.948 mM | 37.8 | μl |
| Solution of polysorbate 20 at 1 mM | 31.2 | μl |
| Commercial solution of Humalog ® | 7.731 | ml |

The final pH was adjusted to 7.4±0.4. The solution was filtered through a 0.22 μm membrane and the clear solution was stored at 4° C.

### I.A17. Preparation of an Insulin Lispro Solution at 100 IU/Ml in the Presence of 9.3 mM Citric Acid Salt and 4 μM Polysorbate 20

For a final volume of 7.8 ml of formulation with a concentration of 9.3 mM citric acid sodium salt and 4 μM polysorbate 20, the various reagents were added up in the amounts specified below and in the following order:

| | | |
|---|---|---|
| Solution of sodium citrate at 0.948 mM | 76.5 | μl |
| Solution of polysorbate 20 at 1 mM | 31.2 | μl |
| Commercial solution of Humalog ® | 7.692 | ml |

The final pH was adjusted to 7.4±0.4. The solution was filtered through a 0.22 μm membrane and the clear solution was stored at 4° C.

### I.A18. Preparation of an Insulin Lispro Solution at 100 IU/Ml in the Presence of 18.6 mM Citric Acid Salt and 4 μM Polysorbate 20

For a final volume of 7.8 ml of formulation with a concentration of 18.6 mM citric acid sodium salt and 4 μM polysorbate 20, the various reagents were added up in the amounts specified below and in the following order:

| | | |
|---|---|---|
| Solution of sodium citrate at 0.948 mM | 153 | μl |
| Solution of polysorbate 20 at 1 mM | 31.2 | μl |
| Commercial solution of Humalog ® | 7.616 | ml |

The final pH was adjusted to 7.4±0.4. The solution was filtered through a 0.22 μm membrane and the clear solution was stored at 4° C.

### I.A19. Preparation of an Insulin Lispro Solution at 100 IU/Ml in the Presence of 4 μM Polysorbate 20

For a final volume of 7.8 ml of formulation with a concentration of 4 μM polysorbate 20, the various reagents were added up in the amounts specified below and in the following order:

| | | |
|---|---|---|
| Solution of polysorbate 20 at 1 mM | 31.2 | μl |
| Commercial solution of Humalog ® | 7.769 | ml |

The final pH was adjusted to 7.4±0.4. The solution was filtered through a 0.22 μm membrane and the clear solution was stored at 4° C.

### I.A20. Preparation of an Insulin Lispro Solution at 100 IU/Ml in the Presence of 9.3 mM Citric Acid Salt and 8 μM Polysorbate 20

For a final volume of 7.8 ml of formulation with a concentration of 9.3 mM citric acid sodium salt and 4 μM polysorbate 20, the various reagents were added up in the amounts specified below and in the following order:

| | | |
|---|---|---|
| Solution of sodium citrate at 0.948 mM | 76.5 | μl |
| Solution of polysorbate 20 at 1 mM | 62.4 | μl |
| Commercial solution of Humalog ® | 7.661 | ml |

The final pH was adjusted to 7.4±0.4. The solution was filtered through a 0.22 μm membrane and the clear solution was stored at 4° C.

### I.B Pharmacodynamics and Pharmacokinetics

### I.B1: Protocol for the Measurement of Pharmacodynamics and Pharmacokinetics of Insulin Solutions

Domestic pigs weighing approximately 50 kg, previously catheterized at the jugular, were fasted 2.5 hours before the start of the experiment. In the hour before the injection of insulin, 3 blood samples were taken to determine the basal level of glucose and insulin.

Injection of insulin at a dose of 0.125 IU/kg for insulin lispro was performed subcutaneously in the flank of the animal using an insulin pen (Novo, Sanofi or Lilly) equipped with a 31 G needle.

Blood samples were then taken every 4 minutes for 20 minutes and then every 10 minutes for up to 3 hours. After each sampling, the catheter was rinsed with a dilute solution of heparin.

A drop of blood was taken to determine glycemia by means of a glucose meter.

The pharmacodynamic curves of the glucose expressed as a percentage of the basal level were then plotted. The following pharmacodynamic endpoint were determined based on standard non-compartmental methods using Phoenix WinNonlin:
Tmin glucose corresponding to the times to the minimum glucose level in the blood
Early T50% Rmin glucose corresponding to the times to 50% of the minimum glucose level in the blood before Tmin
AUC-BG0-30 min corresponding to the early partial areas below the baseline and above the glucodynamic response curve from time 0 to time 30 minutes

T50% Rmin glucose and Tmin glucose are commonly used to evaluate the onset of glucose-lowering effect of insulin formulations. AUC-BG0-30 min is commonly used to evaluate the early glucose lowering effect of insulin formulations. Early T50% Rmin glucose were estimated by linear interpolation.

The remaining blood was collected in a dry tube and centrifuged to isolate the serum. Insulin levels in serum samples were measured by the sandwich ELISA (Enzyme-Linked ImmunoSorbent Assay) method for each pig.

The pharmacokinetic curves expressed in delta of the basal level were then plotted. The following pharmacokinetic parameters were determined based on standard non-compartmental methods using Phoenix WinNonlin:
Tmax insulin corresponding to the time to reach the maximum serum insulin concentration
Early T20% Cmax insulin corresponding to the time to reach 20% of the maximum serum insulin concentration before Tmax
Early T50% Cmax insulin corresponding to the time to reach 50% of the maximum serum insulin concentration before Tmax
AUC-Ins0-15 min corresponding to the early partial areas under the serum insulin curve from time 0 to time 15 minutes post-administration
AUC-Ins0-30 min corresponding to the early partial areas under the serum insulin curve from time 0 to time 30 minutes post-administration

Early T50% Cmax insulin and Tmax insulin are commonly used to evaluate the onset of exposure of insulin formulations. AUC-Ins0-15 min and AUC-Ins0-30 min are commonly used to evaluate the early exposure of insulin formulations.

### I.B2: Pharmacodynamic and Pharmacokinetic Results of Insulin Solutions of Examples I.A1 and I.A4

| Example | Insulin | Sodium citrate | Number of pigs |
|---|---|---|---|
| I.A1 | lispro | — | 9 |
| I.A4 | lispro | 9.3 mM | 12 |

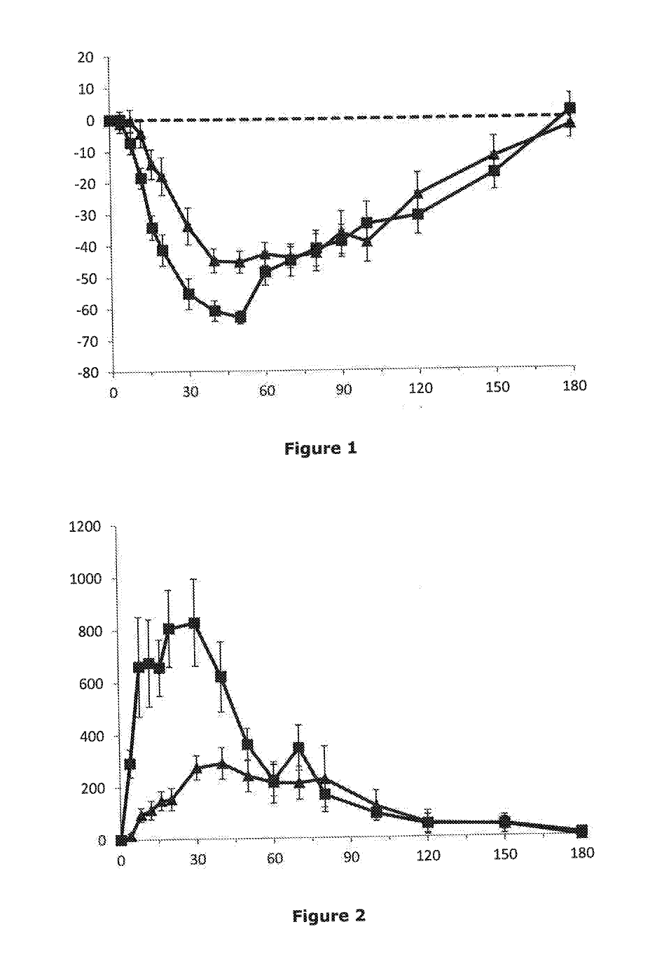

The pharmacodynamic results obtained with the compositions described in Examples I.A1 and I.A4 are shown in FIG. 1. Analysis of these curves shows that the composition comprising 9.3 mM citric acid salt of Example I.A4 (curve plotted with the squares corresponding to Example I.A4) induces a faster action than that of the Humalog® commercial composition of Example I.A1 (curve plotted with the triangles corresponding to Example I.A1). Pharmacodynamic parameters were reported in the table below:

| | | Early T50% | AUC-BG0- |
|---|---|---|---|
| | Tmin glucose | Rmin glucose | 30 min |
| Example | (min) | (min) | (min*% BL) |
| I.A1 | 61 ± 23 | 26 ± 9 | 398 ± 266 |
| I.A4 | 46 ± 13 | 18 ± 6 | 803 ± 350 |

The pharmacokinetic results obtained with the compositions described in Examples I.A1 and I.A4 are shown in FIG. 2. Analysis of these curves shows that the composition of Example I.A4 comprising 9.3 mM citric acid salt (curve plotted with the squares corresponding to Example I.A4) induces a faster absorption of insulin lispro than that of the Humalog® commercial composition of Example I.A1 (curve plotted with the triangles corresponding to Example I.A1). Pharmacokinetic parameters are reported in the table below:

| | | Early T50% | AUC-Ins0- | AUC-Ins0- |
|---|---|---|---|---|
| | Tmax insulin | Cmax insulin | 15 min | 30 min |
| Example | (min) | (min) | (min*mU/L) | (min*mU/L) |
| I.A1 | 39 ± 18 | 24 ± 14 | 1041 ± 750 | 3927 ± 2003 |
| I.A4 | 23 ± 10 | 10 ± 5 | 7132 ± 5403 | 18888 ± 11929 |

### B3: Pharmacokinetic and Pharmacokinetic Results of the Insulin Solutions of Examples I.A1 and I.A9

| | | | Number |
|---|---|---|---|
| Example | Insulin | Sodium citrate | of pigs |
| I.A1 | lispro | — | 9 |
| I.A9 | lispro | 18.6 mM | 8 |

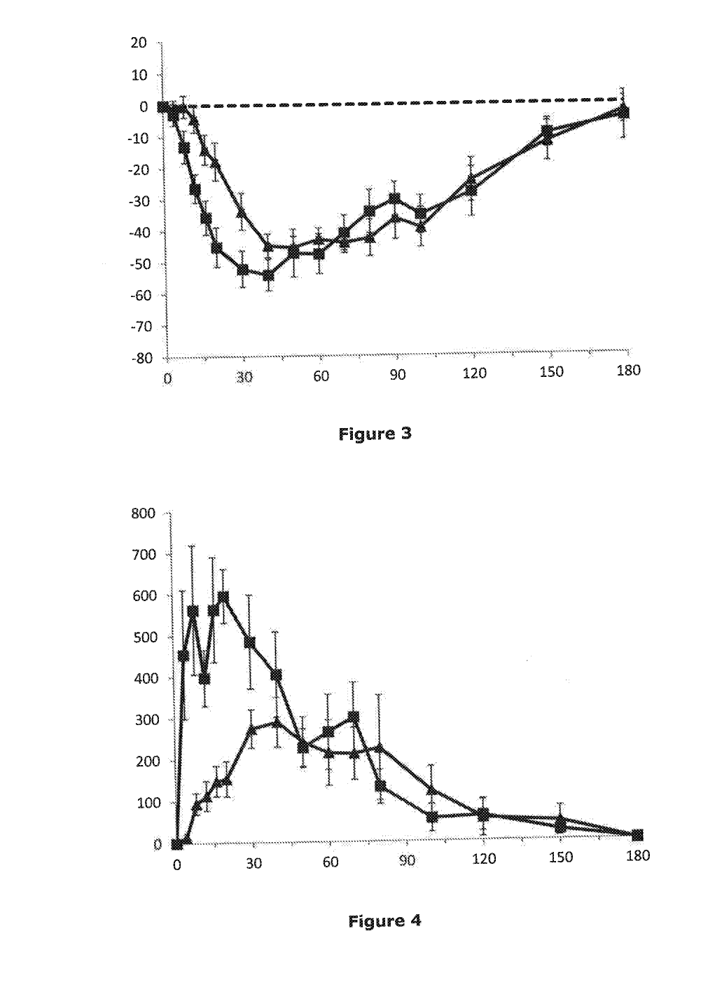

The pharmacodynamic results obtained with the compositions described in Examples I.A1 and I.A9 are shown in FIG. 3. Analysis of these curves shows that the composition of Example I.A9 comprising 18.6 mM citric acid salt (curve plotted with the squares corresponding to Example I.A9) induces a faster action than that of the Humalog® commercial composition of Example I.A1 (curve plotted with the triangles corresponding to Example I.A1). Pharmacodynamic parameters are reported in the table below:

| | | Early T50% | AUC-BG0- |
|---|---|---|---|
| | Tmin glucose | Rmin glucose | 30 min |
| Example | (min) | (min) | (min*% BL) |
| I.A1 | 61 ± 23 | 26 ± 9 | 398 ± 266 |
| I.A9 | 40 ± 12 | 14 ± 4 | 887 ± 359 |

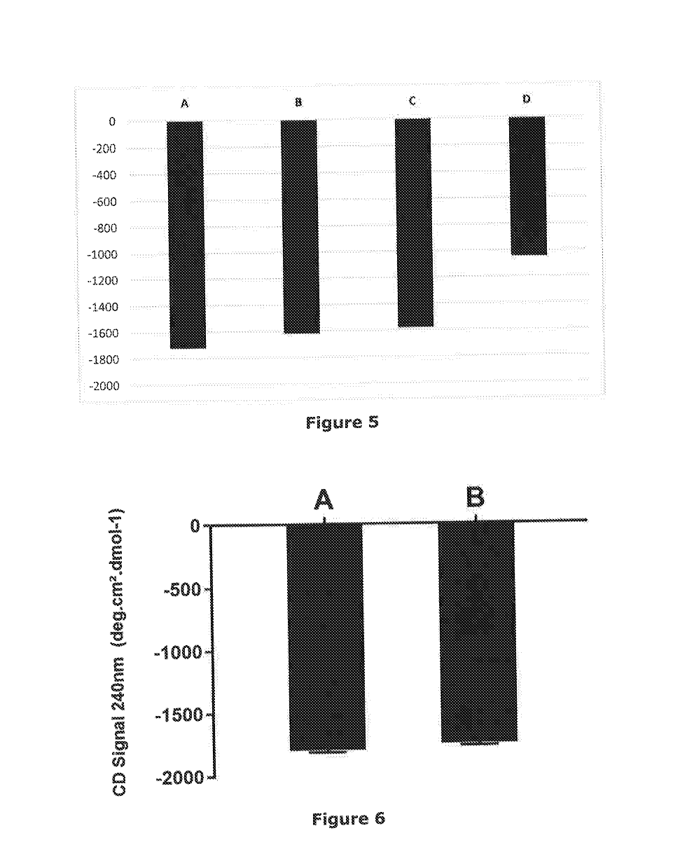

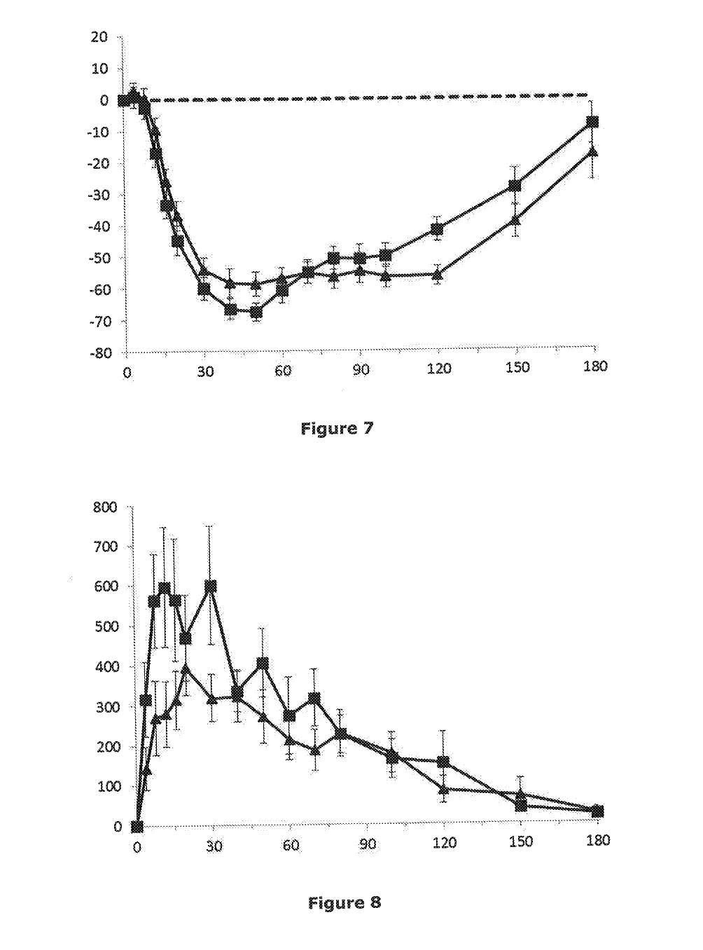

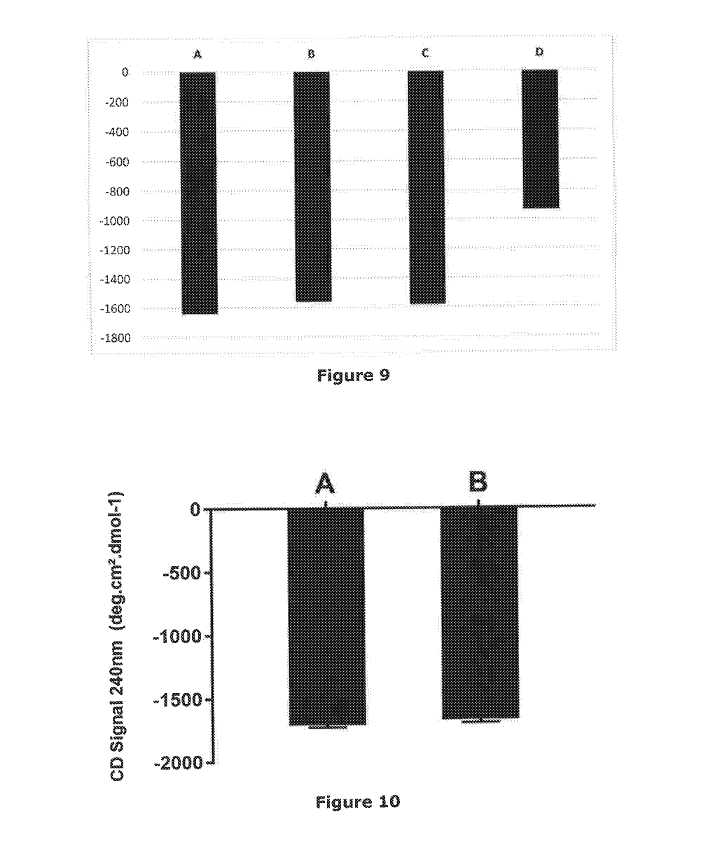

The pharmacokinetic results obtained with the compositions described in Examples I.A1 and I.A9 are shown in FIG. 4. Analysis of these curves shows that the composition of Example I.A9 comprising 18.6 mM citric acid salt (curve plotted with the squares corresponding to Example I.A9) induces a faster absorption of insulin lispro than that of the Humalog® commercial composition of Example I.A1 (curve plotted with the triangles corresponding to Example I.A1).). Pharmacokinetic parameters are reported in the table below:

| | | Early T50% | AUC-Ins0- | AUC-Ins0- |
|---|---|---|---|---|
| | Tmax insulin | Cmax insulin | 15 min | 30 min |
| Example | (min) | (min) | (min*mU/L) | (min*mU/L) |
| I.A1 | 39 ± 18 | 24 ± 14 | 1041 ± 750 | 3927 ± 2003 |
| I.A9 | 24 ± 20 | 5 ± 5 | 6244 ± 4430 | 14501 ± 6811 |

### I.C Circular Dichroism

### I.C1: The Association Status of Insulin Lispro was Evaluated by Circular Dichroism in the Presence of Citric Acid Salt

Circular dichroism allows examining the secondary and quaternary structure of insulin. Insulin monomers organized themselves into dimers and hexamers. The hexamer is the most physically and chemically stable form of insulin. There are two hexameric forms, the R6 form and the T6 form. Insulin lispro has a strong signal at 240 nm, characteristic of the hexameric R6 form (the most stable form). The loss of the signal at 240 nm is related to a destabilization of the hexameric R6 form and the passage from the R6 form to the T6 form.

The results obtained are presented in FIG. 5. FIG. 5 describes the CD signal at 240 nm (deg·cm²·dmol⁻¹) on the ordinate and on the abscissa:

A: insulin lispro 100 IU/ml (Example I.A1)
B: insulin lispro 100 IU/ml and sodium citrate at 9.3 mM (Example I.A4)
C: insulin lispro 100 IU/ml and sodium citrate at 18.6 mM (Example I.A9)
D: insulin lispro 100 IU/ml and EDTA at 300 μM (Example I.A14)

EDTA completely deconstructs the R6 form of insulin lispro. EDTA therefore has a marked effect on the hexamer.

On the contrary, the citric acid salts have little or no impact on the CD signal at 240 nm.

These compounds therefore have given little or no impact on the R6 structure of the hexamer. The insulin lispro in the compositions as described above is therefore considered to be in the hexameric form.

### I.C2: The Association Status of Insulin Lispro was Evaluated by Circular Dichroism in the Presence of Citric Acid Salt and Polysorbate 20.

Circular dichroism has been performed at 240 nm on the following compositions (FIG. 6):

A: insulin lispro 100 IU/ml (Example I.A1)
B: insulin lispro 100 IU/ml, sodium citrate at 9.3 mM and Polysorbate at 8 μM (Example I.A20)

The citric acid salts in combination with polysorbate 20 have little or no impact on the CD signal at 240 nm.

These compounds therefore have given little or no impact on the R6 structure of the hexamer. The insulin lispro in the compositions as described above was therefore considered to be in the hexameric form.

### I.D Physical Stability Studies

Formulations I.A19, I.A15, I.A16, I.A4, I.A17, I.A9, and I.A18 were added (1 ml) to 2 ml Schott type vial. Five replicates were done for each formulation.

The vials were placed 6 days on an horizontal stirrer POS-300Grant-Bio (50-300 rpm; orbit: 10 mm) in an oven at 25° C. and at a speed of 250 rpm. Visual inspection was done every 2 days. Vials containing a clear and colorless solution with no aggregate are considered as a “pass”, otherwise it was considered as a “fail”. Pass percentages are given in the following table.

| Compositions | 0 days | 2 days | 4 days | 6 days |
|---|---|---|---|---|
| Humalog ® (insulin lispro 100 | 100 | 60 | 40 | 0 |
| U/ml) | | | | |
| IA19 (insulin lispro + PS20 4 μM) | 100 | 100 | 100 | 100 |
| IA15 (insulin lispro + Citrate 4.6 | 100 | 0 | 0 | 0 |
| mM) | | | | |
| IA16 (insulin lispro + Citrate 4.6 | 100 | 100 | 100 | 80 |
| mM + PS20 4 μM) | | | | |
| IA4 (insulin lispro + Citrate 9.3 | 100 | 0 | 0 | 0 |
| mM) | | | | |
| IA17 (insulin lispro + Citrate 9.3 | 100 | 100 | 100 | 100 |
| mM + PS20 4 μM) | | | | |
| IA9 (insulin lispro + Citrate | 100 | 0 | 0 | 0 |
| 18.6 mM) | | | | |
| IA18 (insulin lispro + Citrate | 100 | 100 | 100 | 80 |
| 18.6 mM + PS20 4 μM) | | | | |

As shown above, in some cases, the citrate alone may lead to the formation of aggregates over time in the solution.

These results show clearly the beneficial impact of the surfactant on the physical stability of the compositions.

II. Insulin Aspart

### Part II.A: Preparing Solutions

### II.A1. Novolog®/Novorapid® Fast-Acting Analog Insulin Solution at 100 U/Ml

This solution is a commercial solution of insulin aspart from Novo Nordisk sold under the name of Novolog®/Novorapid®. This product is a fast-acting analog insulin.

Novolog®/Novorapid® U100 has the following composition: glycerin (16 mg/ml), phenol (1.5 mg/ml), meta-cresol (1.72 mg/ml), zinc (19.6 μg/ml), disodium hydrogen phosphate dihydrate (1.25 mg/ml), sodium chloride (0.58 mg/ml), and water for injection. Novolog® has a pH of 7.2 to 7.6. HCl (10%) and/or NaOH (10%) may be added to adjust pH. (Novolog FDA label (ref ID 3212914) https://www.accessdata.fda.gov/drugsatfda_docs/label/2012/020986s057lbl.pdf)

### II.A2. Preparation of an Insulin Aspart Solution at 200 U/Ml.

The commercial formulation of insulin aspart (Novolog®/Novorapid®) at 100 U/ml was concentrated by using Amicon Ultra-15 centrifuge tubes with a 3 kDa cut-off. The Amicon tubes were first rinsed with 12 ml of deionized water. 12 ml of the commercial formulation were centrifuged for 35 minutes at 4000 g at 20° C. The volume of the retentate was measured and the concentration thus estimated. All retentates were pooled and the overall concentration was estimated (>200 U/ml).

The concentration of this concentrated insulin aspart solution was adjusted to 200 U/ml by the addition of the commercial formulation of insulin aspart (Novolog®/Novorapid®). The concentrated formulation of insulin aspart has the same concentrations of excipients (m-cresol, glycerin, phosphate, phenol and sodium chloride) as the commercial formulation at 100 U/ml.

The final pH was adjusted to 7.4±0.4. The clear solution was filtered through a 0.22 μm membrane and stored at 4° C.

### II.A3. Preparation of an Insulin Aspart Solution at 300, 400 and 500 U/Ml

Concentrated formulations of insulin aspart at 300 U/ml, 400 U/ml or 500 U/ml (and at all intermediate concentrations) are prepared on the basis of the protocol of Example II.A2 relating to the preparation of an insulin aspart solution at 200 U/ml. The commercial formulation of insulin was concentrated using Amicon Ultra-15 centrifuge tubes with a 3 kDa cut-off. The Amicon tubes are first rinsed with 12 ml of deionized water. 12 ml of the commercial formulation are centrifuged at 4000 g and 20° C. By controlling the centrifugation time, it was possible to adjust the final insulin concentration in the formulation. The volume of the retentate was measured and the concentration thus estimated. All retentates are pooled and the overall concentration was estimated (>300, 400 or 500 U/ml).

The concentration of this concentrated insulin solution was adjusted to the desired concentration (e.g. 300 U/ml, 400 U/ml or 500 U/ml) by adding the commercial formulation of insulin aspart (Novolog®/Novorapid®). The concentrated insulin formulation has the same excipient concentrations as the commercial formulation at 100 U/ml.

The final pH was adjusted to 7.4±0.4. The clear solution was filtered through a 0.22 μm membrane and stored at 4° C.

### II.A4. Preparation of an Insulin Aspart Solution at 100 U/Ml in the Presence of 9.3 mM Citric Acid Salt

For a final volume of 100 ml of composition with a concentration of 9.3 mM citric acid salt, the various reagents are added up in the amounts specified below and in the following order:

| | | |
|---|---|---|
| Sodium citrate | 240 | mg |
| Commercial solution of Novolog ®/Novorapid ® | 100 | ml |

The final pH was adjusted to 7.4±0.4. The clear solution was filtered through a 0.22 μm membrane and stored at 4° C.

### II.A5. Preparation of an Insulin Aspart Solution at 200 U/Ml in the Presence of 18.6 mM Citric Acid Salt

For a final volume of 100 ml of formulation with a concentration of 18.6 mM citric acid salt, the various reagents are added up in the amounts specified below and in the following order:

| | | |
|---|---|---|
| Sodium citrate | 480 | mg |
| Insulin aspart at 200 U/ml | 100 | ml |

The final pH was adjusted to 7.4±0.4. The clear solution was filtered through a 0.22 μm membrane and stored at 4° C.

### II.A6. Preparation of an Insulin Aspart Solution at 300 U/Ml in the Presence of 27.9 mM Citric Acid Salt.

For a final volume of 100 ml of formulation with a concentration of 27.9 mM citric acid salt, the various reagents are added up in the amounts specified below and in the following order:

| | | |
|---|---|---|
| Sodium citrate | 720 | mg |
| Insulin aspart at 300 U/ml | 100 | ml |

The final pH was adjusted to 7.4±0.4. The clear solution was filtered through a 0.22 μm membrane and stored at 4° C.

### II.A7. Preparation of an Insulin Aspart Solution at 400 U/Ml in the Presence of 37.2 mM Citric Acid Salt.

For a final volume of 100 ml of formulation with a concentration of 37.2 mM citric acid salt, the various reagents are added up in the amounts specified below and in the following order:

| | | |
|---|---|---|
| Sodium citrate | 960 | mg |
| Insulin aspart at 400 U/ml | 100 | ml |

The final pH was adjusted to 7.4±0.4. The clear solution was filtered through a 0.22 μm membrane and stored at 4° C.

### II.A8. Preparation of an Insulin Aspart Solution at 500 U/Ml in the Presence of 46.5 mM Citric Acid Salt

For a final volume of 100 ml of formulation with a concentration of 46.5 mM citric acid salt, the various reagents are added up in the amounts specified below and in the following order:

| | | |
|---|---|---|
| Sodium citrate | 1200 | mg |
| Insulin aspart at 500 U/ml | 100 | ml |

The final pH was adjusted to 7.4±0.4. The clear solution was filtered through a 0.22 μm membrane and stored at 4° C.

### II.A9. Preparation of an Insulin Aspart Solution at 100 U/Ml in the Presence of 18.6 mM Citric Acid Salt

For a final volume of 100 ml of formulation with a concentration of 18.6 mM citric acid salt, the various reagents are added up in the amounts specified below and in the following order:

| | | |
|---|---|---|
| Sodium citrate | 480 | mg |
| Commercial solution of Humalog ® | 100 | ml |

The final pH was adjusted to 7.4±0.4. The clear solution was filtered through a 0.22 μm membrane and stored at 4° C.

### II.A10. Preparation of an Insulin Aspart Solution at 200 U/Ml in the Presence of 37.2 mM Citric Acid Salt

For a final volume of 100 ml of formulation with a concentration of 37.2 mM citric acid salt, the various reagents are added up in the amounts specified below and in the following order:

| | | |
|---|---|---|
| Sodium citrate | 960 | mg |
| Insulin aspart at 200 U/ml | 100 | ml |

The final pH was adjusted to 7.4±0.4. The clear solution was filtered through a 0.22 μm membrane and stored at 4° C.

### II.A11. Preparation of an Insulin Aspart Solution at 300 U/Ml in the Presence of 55.8 mM Citric Acid Salt

For a final volume of 100 ml of formulation with a concentration of 55.8 mM citric acid salt, the various reagents are added up in the amounts specified below and in the following order:

| | | |
|---|---|---|
| Sodium citrate | 1440 | mg |
| Insulin aspart at 300 U/ml | 100 | ml |

The final pH was adjusted to 7.4-0.4. The clear solution was filtered through a 0.22 μm membrane and stored at 4° C.

### II.A12. Preparation of an Insulin Aspart Solution at 400 U/Ml in the Presence of 74.4 mM Citric Acid Salt

For a final volume of 100 ml of formulation with a concentration of 74.4 mM citric acid salt, the various reagents are added up in the amounts specified below and in the following order:

| | | |
|---|---|---|
| Sodium citrate | 1920 | mg |
| Insulin aspart at 400 U/ml | 100 | ml |

The final pH was adjusted to 7.4±0.4. The clear solution was filtered through a 0.22 μm membrane and stored at 4° C.

### II.A13. Preparation of an Insulin Aspart Solution at 500 U/Ml in the Presence of 93.0 mM Citric Acid Salt

For a final volume of 100 ml of formulation with a concentration of 93.0 mM citric acid salt, the various reagents are added up in the amounts specified below and in the following order:

| | | |
|---|---|---|
| Sodium citrate | 2400 | mg |
| Insulin aspart at 500 U/ml | 100 | ml |

The final pH was adjusted to 7.4±0.4. The clear solution was filtered through a 0.22 μm membrane and stored at 4° C.

### II.A14. Preparation of an Insulin Aspart Solution at 100 U/Ml in the Presence of EDTA at 300 μM

For a final volume of 100 ml of formulation with a concentration of 300 μM EDTA, the various reagents are added up in the amounts specified below and in the following order:

| | | |
|---|---|---|
| Commercial solution of Novolog ®/Novorapid ® at 100 U/ml | 100 | ml |
| Commercial solution of EDTA at 0.5M | 60 | μL |

The final pH was adjusted to 7.4±0.4. The clear solution was filtered through a 0.22 μm membrane and stored at 4° C.

### II.A15. Preparation of an Insulin Aspart Solution at 100 IU/Ml in the Presence of 4.6 mM Citric Acid Salt

For a final volume of 7.8 ml of formulation with a concentration of 4.6 mM citric acid sodium salt, the various reagents are added up in the amounts specified below and in the following order:

| | | |
|---|---|---|
| Solution of sodium citrate at 0.948 mM | 37.8 | μl |
| Commercial solution of Novolog ®/Novorapid ® | 7.769 | ml |

The final pH was adjusted to 7.4±0.4. The solution was filtered through a 0.22 μm membrane and the clear solution was stored at 4° C.

### II.A16. Preparation of an Insulin Aspart Solution at 100 IU/Ml in the Presence of 4.6 mM Citric Acid Salt and 4 μM Polysorbate 20 (or PS20)

For a final volume of 7.8 ml of formulation with a concentration of 4.6 mM citric acid sodium salt and 4 μM polysorbate 20 (CAS 9005-64-5), the various reagents are added up in the amounts specified below and in the following order:

| | | |
|---|---|---|
| Solution of sodium citrate at 0.948 mM | 37.8 | μl |
| Solution of polysorbate 20 at 1 mM | 31.2 | μl |
| Commercial solution of Novolog ®/Novorapid ® | 7.731 | ml |

The final pH was adjusted to 7.4±0.4. The solution was filtered through a 0.22 μm membrane and the clear solution was stored at 4° C.

### II.A17. Preparation of an Insulin Aspart Solution at 100 IU/Ml in the Presence of 9.3 mM Citric Acid Salt and 4 μM Polysorbate 20

For a final volume of 7.8 ml of formulation with a concentration of 9.3 mM citric acid sodium salt and 4 μM polysorbate 20, the various reagents are added up in the amounts specified below and in the following order:

| | | |
|---|---|---|
| Solution of sodium citrate at 0.948 mM | 76.5 | μl |
| Solution of polysorbate 20 at 1 mM | 31.2 | μl |
| Commercial solution of Novolog ®/Novorapid ® | 7.692 | ml |

The final pH was adjusted to 7.4±0.4. The solution was filtered through a 0.22 μm membrane and the clear solution was stored at 4° C.

### II.A18. Preparation of an Insulin Aspart Solution at 100 IU/Ml in the Presence of 18.6 mM Citric Acid Salt and 4 μM Polysorbate 20

For a final volume of 7.8 ml of formulation with a concentration of 18.6 mM citric acid sodium salt and 4 μM polysorbate 20, the various reagents are added up in the amounts specified below and in the following order:

| | | |
|---|---|---|
| Solution of sodium citrate at 0.948 mM | 153 | μl |
| Solution of polysorbate 20 at 1 mM | 31.2 | μl |
| Commercial solution of Novolog ®/Novorapid ® | 7.616 | ml |

The final pH was adjusted to 7.4±0.4. The solution was filtered through a 0.22 μm membrane and the clear solution was stored at 4° C.

### II.A19. Preparation of an Insulin Aspart Solution at 100 IU/Ml in the Presence of 4 μM Polysorbate 20

For a final volume of 7.8 ml of formulation with a concentration of 4 μM polysorbate 20, the various reagents are added up in the amounts specified below and in the following order:

| | | |
|---|---|---|
| Solution of polysorbate 20 at 1 mM | 31.2 | μl |
| Commercial solution of Humalog ® | 7.769 | ml |

The final pH was adjusted to 7.4±0.4. The solution was filtered through a 0.22 μm membrane and the clear solution was stored at 4° C.

### II.A20. Preparation of an Insulin Aspart Solution at 100 IU/Ml in the Presence of 9.3 mM Citric Acid Salt and 8 μM Polysorbate 20

For a final volume of 7.8 ml of formulation with a concentration of 9.3 mM citric acid sodium salt and 4 μM polysorbate 20, the various reagents are added up in the amounts specified below and in the following order:

| | | |
|---|---|---|
| Solution of sodium citrate at 0.948 mM | 76.5 | μl |
| Solution of polysorbate 20 at 1 mM | 62.4 | μl |
| Commercial solution of Novolog ®/Novorapid ® | 7.661 | ml |

The final pH was adjusted to 7.4±0.4. The solution was filtered through a 0.22 μm membrane and the clear solution was stored at 4° C.

### II.B Pharmacodynamics and Pharmacokinetics

### II.B1: Protocol for the Measurement of Pharmacodynamics and Pharmacokinetics of Insulin Solutions

Domestic pigs weighing approximately 50 kg, previously catheterized at the jugular, were fasted 2.5 hours before the start of the experiment. In the hour before the injection of insulin, 3 blood samples were taken to determine the basal level of glucose and insulin.

Injection of insulin at a dose of 0.125 IU/kg for insulin lispro was performed subcutaneously in the flank of the animal using an insulin pen (Novo, Sanofi or Lilly) equipped with a 31 G needle.

Blood samples were then taken every 4 minutes for 20 minutes and then every 10 minutes for up to 3 hours. After each sampling, the catheter was rinsed with a dilute solution of heparin.

A drop of blood was taken to determine glycemia by means of a glucose meter.

The pharmacodynamic curves of the glucose expressed as a percentage of the basal level were then plotted. The following pharmacodynamic endpoint were determined based on standard non-compartmental methods using Phoenix WinNonlin:
Tmin glucose corresponding to the times to the minimum glucose level in the blood
Early T50% Rmin glucose corresponding to the times to 50% of the minimum glucose level in the blood before Tmin
AUC-BG0-30 min corresponding to the early partial areas below the baseline and above the glucodynamic response curve from time 0 to time 30 minutes
   Early T50% Rmin glucose and Tmin glucose are commonly used to evaluate the onset of glucose-lowering effect of insulin formulations. AUC-BG0-30 min is commonly used to evaluate the early glucose lowering effect of insulin formulations. Early T50% Rmin glucose were estimated by linear interpolation.

The remaining blood was collected in a dry tube and centrifuged to isolate the serum. Insulin levels in serum samples were measured by the sandwich ELISA (Enzyme-Linked ImmunoSorbent Assay) method for each pig.

The pharmacokinetic curves expressed in delta of the basal level were then plotted. The following pharmacokinetic parameters were determined based on standard non-compartmental methods using Phoenix WinNonlin:
Tmax insulin corresponding to the time to reach the maximum serum insulin concentration
Early T50% Cmax insulin corresponding to the time to reach 50% of the maximum serum insulin concentration before Tmax
AUC-Ins0-15 min corresponding to the early partial areas under the serum insulin curve from time 0 to time 15 minutes post-administration
AUC-Ins0-30 min corresponding to the early partial areas under the serum insulin curve from time 0 to time 30 minutes post-administration

Early T50% Cmax insulin and Tmax insulin are commonly used to evaluate the onset of exposure of insulin formulations. AUC-Ins0-15 min and AUC-Ins0-30 min are commonly used to evaluate the early exposure of insulin formulations. Early T50% Cmax insulin were estimated by linear interpolation.

### II.B2: Pharmacodynamic and Pharmacokinetic Results of Insulin Solutions of Examples II.A1 and II.A4

| Example | Insulin | Sodium citrate | Number of pigs |
|---|---|---|---|
| II.A1 | aspart | — | 12 |
| II.A4 | aspart | 9.3 mM Sodium citrate | 12 |

The pharmacodynamic results obtained with the compositions described in Examples II.A1 and II.A4 are shown in FIG. 7. Analysis of these curves shows that the composition of Example II.A4 comprising the 9.3 mM citric acid salt (curve plotted with the squares corresponding to Example II.A4) induces a faster action than that of the Novolog®/Novorapid® commercial composition of Example II.A1 (curve plotted with the triangles corresponding to Example II.A1). Pharmacodynamic parameters are reported in the table below:

| | | Early T50% | AUC-BG0- |
|---|---|---|---|
| | Tmin glucose | Rmin glucose | 30 min |
| Example | (min) | (min) | (min*% BL) |
| II.A1 | 63 ± 27 | 20 ± 6 | 660 ± 315 |
| II.A4 | 49 ± 16 | 18 ± 4 | 822 ± 352 |

The pharmacokinetic results obtained with the compositions described in Examples II.A1 and II.A4 are shown in FIG. 8. Analysis of these curves shows that the composition of Example II.A4 comprising the 9.3 mM citric acid salt (curve plotted with the squares corresponding to Example II.A4) induces a faster absorption of insulin aspart than that of the Novolog®/Novorapid® commercial composition of Example II.A1 (curve plotted with the triangles corresponding to Example II.A1). Pharmacokinetic parameters are reported in the table below:

| | | Early T50% | AUC-Ins0- | AUC-Ins0- |
|---|---|---|---|---|
| | Tmax insulin | Cmax insulin | 15 min | 30 min |
| Example | (min) | (min) | (min*mU/L) | (min*mU/L) |
| II.A1 | 33 ± 20 | 13 ± 7 | 3093 ± 3338 | 8399 ± 6087 |
| II.A4 | 26 ± 20 | 7 ± 6 | 6460 ± 4690 | 14443 ± 8670 |

### II.C Circular Dichroism

### II.C1: The Association Status of Insulin Aspart was Evaluated by Circular Dichroism in the Presence of Citric Acid Salt

Circular dichroism allows examining the secondary and quaternary structure of insulin. Insulin monomers organized themselves into dimers and hexamers. The hexamer is the most physically and chemically stable form of insulin. There are two hexameric forms, the R6 form and the T6 form. Insulin aspart has a strong signal at 240 nm, characteristic of the hexameric form R6 (the most stable form). The loss of the signal at 240 nm is related to a destabilization of the hexameric form R6 and the passage from R6 form to T6 form.

The results obtained are presented in FIG. 9. FIG. 9 describes the CD signal at 240 nm (deg·cm²·dmol⁻¹) on the ordinate and on the abscissa:

A: insulin aspart 100 U/ml (Example II.A1)
B: insulin aspart 100 U/ml and sodium citrate at 9.3 mM (Example II.A4)
C: insulin aspart 100 U/ml and sodium citrate at 18.6 mM (Example II.A9)
D: insulin aspart 100 U/ml and EDTA at 300 μM (Example II.A14)

EDTA completely deconstructs the R6 form of insulin aspart. EDTA therefore has a marked effect on the hexamer.

On the contrary, the citric acid salts have little or no impact on the CD signal at 240 nm.

These compounds therefore have given little or no impact on the R6 structure of the hexamer. The insulin aspart in the compositions as described above is therefore considered to be in the hexameric form.

### I.C2: The Association Status of Insulin Aspart was Evaluated by Circular Dichroism in the Presence of Citric Acid Salt and Polysorbate 20.

Circular dichroism has been performed at 240 nm on the following compositions (FIG. 10):

A: insulin aspart 100 IU/ml (Example II.A1)
B: insulin aspart 100 IU/ml, sodium citrate at 9.3 mM and Polysorbate at 8 μM (Example II.A20)

The citric acid salts in combination with polysorbate 20 have little or no impact on the CD signal at 240 nm.

These compounds therefore have given little or no impact on the R6 structure of the hexamer. The insulin lispro in the compositions as described above is therefore considered to be in the hexameric form.

1. An aqueous pharmaceutical composition comprising:
insulin in hexameric form, and
citric acid or a salt thereof at a concentration from 6 to 30 mM,
wherein the composition is suitable for injecting into a diabetic patient to treat diabetes,
wherein the composition does not contain any one of
EDTA,
anionic compounds whose chemical structure includes at least one carboxyl functional group to which is covalently bonded an amino-acid residue,
saccharides whose chemical structure includes at least one carboxyl functional group to which is covalently bonded an amino-acid residue, and
oligosaccharides whose chemical structure includes at least one carboxyl functional group to which is covalently bonded an amino-acid residue.

2. An aqueous pharmaceutical composition comprising:
insulin in hexameric form, and citric acid or a salt thereof at a concentration from 6 to 30 mM,
wherein the composition is suitable for injecting into a diabetic patient to treat diabetes, and
wherein the composition does not contain more than 80 μg/ml of an insulin accelerant either alone or in combination with citrate.

3. The aqueous pharmaceutical composition according to claim 1, wherein the onset of action is at least 10% more rapid than that of a reference composition at the same insulin concentration in absence of citric acid or a salt thereof when measured according to the protocol for the measurement of pharmacodynamics and pharmacokinetics of insulin solutions.

4. The aqueous pharmaceutical composition according to claim 1, wherein the onset of appearance is at least 10% more rapid than that of a reference composition at the same insulin concentration in absence of citric acid or a salt thereof when measured according to the protocol for the measurement of pharmacodynamics and pharmacokinetics of insulin solutions.

5. The aqueous pharmaceutical composition according to claim 1, wherein the fast uptake of insulin is at least 10% more than that of a reference composition at the same insulin concentration in absence of citric acid or a salt thereof when measured according to the protocol for the measurement of pharmacodynamics and pharmacokinetics of insulin solutions.

6. The aqueous pharmaceutical composition according to claim 1, wherein the insulin is insulin lispro or insulin aspart.

7. The aqueous pharmaceutical composition according to claim 1, wherein the insulin is insulin lispro.

8. The aqueous pharmaceutical composition according to claim 1, wherein the insulin concentration in the composition is between 600 to 1800 μM (100 to 300 U/ml).

9. The aqueous pharmaceutical composition according to claim 1, wherein the composition further comprises a surfactant.

10. The aqueous pharmaceutical composition according to claim 1, wherein the pH of the composition is from 6.0 to 8.0.

11. An injection device which contains the aqueous pharmaceutical composition according to claim 1.

12. A vial which contains the aqueous pharmaceutical composition according to claim 1.

13. A cartridge adapted for being inserted into an injection device and comprising the aqueous pharmaceutical composition according to claim 1.

14. A unit dose which contains the aqueous pharmaceutical composition according to claim 1.

15. A method of treating diabetes comprising administering to a human patient in need thereof an effective dose of the aqueous pharmaceutical composition according to claim 1.

16. A method according to claim 14, wherein the administering produces an uptake of insulin or an onset of appearance in the patient's blood that is at least 10% more rapid than that for a reference composition with the same concentration of insulin that does not contain the citric acid or salt thereof.
